SEQUENCE LISTING (13 sequences)

SEQ ID NO: 1 - DNA, 8980 bp, Homo sapiens
ggaggttgtggggccgccgccgcggagcaccgtccccgccgccgcccgagcccgagcccgagcccgcgcacccgcccgcgccgccgccgccgccgcccgaacagcctcccagcctgggcccccggcggcgccgtggccgcgtcccggctgtcgccgcccgagcccgagcccgcgcgccggcgggtggcggcgcaggctgaggagatgcggcgcggagcgccggagcagggctagagccggccgccgccgcccgccgcggtaagcgcagccccggcccggcgcccgcgggccattgtccgccgcccgccccgcgccccgcgcagcctgcaggccttggagcccgcggcaggtggacgccgccggtccacacccgccccgcgcgcggccgtgggaggcgggggccagcgctggccgcgcgccgtgggacccgccggtccccagggccgcccggccccttctggacctttccacccgcgccgcgaggcggcttcgcccgccggggcgggggcgcgggggtgggcacggcaggcagcggcgccgtctcccggtgcggggcccgcgccccccgagcaggttcatctgcagaagccagcggacgcctctgttcaacttgtgggttacctggctcatgagaccttgccggcgaggctcggcgcttgaacgtctgtgacccagccctcaccgtcccggtacttgtatgtgttggtgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacgccgagagactcacccttcccgccctccagcagaggctctcccttttccccggcacccacctcactccctacctgagcacctcgcccttggagcgggacggaggggcagcgcacagccctcttctgcagcacatggtcttactggagcagccgccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccccagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacaggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaaggagcctgtggcaacaggcggccgagctgccgaattcagttgacacgaggcacagaaaacaaatatcaaagatctaataatacaaaacaaacttgattaaaactggtgcttaaagtttattacccacaactccacagtctctgtgtaaaccactcgactcatcttgtagcttatttttttttaaagaggacgttttctacggctgtggcccgcctctgtgaaccatagcggtgtgcggcggggggtctgcacccgggtgggggacagagggacctttaaagaaaacaaaactggacagaaacaggaatgtgagctgggggagctggcttgagtttctcaaaagccatcggaagatgcgagtttgtgcctttttttttattgctctggtggatttttgtggctgggttttctgaagtctgaggaacaatgccttaagaaaaaacaaacagcaggaatcggtgggacagtttcctgtggccagccgagcctggcagtgctggcaccgcgagctggcctgacgcctcaagcacgggcaccagccgtcatctccggggccaggggctgcagcccggcggtccctgttttgctttattgctgtttaagaaaaatggaggtagttccaaaaaagtggcaaatcccgttggaggttttgaagtccaacaaattttaaacgaatccaaagtgttctcacacgtcacatacgattgagcatctccatctggtcgtgaagcatgtggtaggcacacttgcagtgttacgatcggaatgctttttattaaaagcaagtagcatgaagtattgcttaaattttaggtataaataaatatatatatgtataatatatattccaatgtattccaagctaagaaacttacttgattcttatgaaatcttgataaaatatttataatgcatttatagaaaaagtatatatatatatataaaatgaatgcagattgcgaaggtccctgcaaatggatggcttgtgaatttgctctcaaggtgcttatggaaagggatcctgattgattgaaattcatgttttctcaagctccagattggctagatttcagatcgccaacacattcgccactgggcaactaccctacaagtttgtactttcattttaattattttctaacagaaccgctcccgtctccaagccttcatgcacatatgtacctaatgagtttttatagcaaagaatataaatttgctgttgatttttgtatgaattttttcacaaaaagatcctgaataagcattgttttatgaattttacatttttcctcaccatttagcaattttctgaatggtaataatgtctaaatctttttcctttctgaattcttgcttgtacatttttttttacctttcaaaggtttttaattatttttgtttttatttttgtacgatgagttttctgcagcgtacagaattgttgctgtcagattctattttcagaaagtgagaggagggaccgtaggtcttttcggagtgacaccaacgattgtgtctttcctggtctgtcctaggagctgtataaagaagcccaggggctctttttaactttcaacactagtagtattacgaggggtggtgtgtttttcccctccgtggcaagggcagggagggttgcttaggatgcccggccaccctgggaggcttgccagatgccgggggcagtcagcattaatgaaactcatgtttaaacttctctgaccacatcgtcaggatagaattctaacttgagttttccaaagaccttttgagcatgtcagcaatgcatggggcacacgtggggctctttacccacttgggtttttccactgcagccacgtggccagccctggattttggagcctgtggctgcaaggaacccagggacccttgttgcctggtgaacctgcagggagggtatgattgcctgaccaggacagccagtctttactctttttctcttcaacagtaactgacagtcacgttttactggtaacttattttccagcacatgaagccaccagtttcattccaaagtgtatattgggttcagacttgggggcagaagttcagacacaccgtgctcaggagggacccagagccgagtttcggagtttggtaaagtttacagggtagcttctgaaattaactcaaacttttgaccaaatgagtgcagattcttggattcacttggtcactgggctgctgatggtcagctctgagacagtggtttgagagcaggcagaacggtcttgggacttgtttgactttcccctccctggtggccactctttgctctgaagcccagattggcaagaggagctggtccattccccattcatggcacagagcagtggcagggcccagctagcaggctcttctggcctccttggcctcattctctgcatagccctctggggatcctgccacctgccctcttaccccgccgtggcttatggggaggaatgcatcatctcactttttttttttaagcagatgatgggataacatggactgctcagtggccaggttatcagtggggggacttaattctaatctcattcaaatggagacgccctctgcaaaggcctggcagggggaggcacgtttcatctgtcagctcactccagcttcacaaatgtgctgagagcattactgtgtagccttttctttgaagacacactcggctcttctccacagcaagcgtccagggcagatggcagaggatctgcctcggcgtctgcaggcgggaccacgtcagggagggttccttcatgtgttctccctgtgggtccttggacctttagcctttttcttcctttgcaaaggccttgggggcactggctgggagtcagcaagcgagcactttatatccctttgagggaaaccctgatgacgccactgggcctcttggcgtctgccctgccctcgcggcttcccgccgtgccgcagcgtgcccacgtgcccacgccccaccagcaggcggctgtcccggaggccgtggcccgctgggactggccgcccctccccagcgtcccagggctctggttctggagggccactttgtcaaggtgtttcagtttttctttacttcttttgaaaatctgtttgcaaggggaaggaccatttcgtaatggtctgacacaaaagcaagtttgatttttgcagcactagcaatggactttgttgtttttctttttgatcagaacattccttctttactggtcacagccacgtgctcattccattcttctttttgtagactttgggcccacgtgttttatgggcattgatacatatataaatatatagatataaatatatatgaatatatttttttaagtttcctacacctggaggttgcatggactgtacgaccggcatgactttatattgtatacagattttgcacgccaaactcggcagctttggggaagaagaaaaatgcctttctgttcccctctcatgacatttgcagatacaaaagatggaaatttttctgtaaaacaaaaccttgaaggagaggagggcggggaagtttgcgtcttattgaacttattcttaagaaattgtactttttattgtaagaaaaataaaaaggactacttaaacatttgtcatattaagaaaaaaagtttatctagcacttgtgacataccaataatagagtttattgtatttatgtggaaacagtgttttagggaaactactcagaattcacagtgaactgcctgtctctctcgagttgatttggaggaattttgttttgttttgttttgtttgtttccttttatctccttccacgggccaggcgagcgccgcccgccctcactggccttgtgacggtttattctgattgagaactgggcggactcgaaagagtccccttttccgcacagctgtgttgactttttaattacttttaggtgatgtatggctaagatttcactttaagcagtcgtgaactgtgcgagcactgtggtttacaattatactttgcatcgaaaggaaaccatttcttcattgtaacgaagctgagcgtgttcttagctcggcctcactttgtctctggcattgattaaaagtctgctattgaaagaaaaagaaagcgaacagtttttgtttgttttttttgccgtgtgtgctccatagtggaggcgctattttccaattgatgagaatgacaaacatatataatctatctatctatctatctatctatctatctatacagggggcttgaaccttactcacccaagagcttcttacggaatgtggtagaaaaccaagttgtaacgacactgtaacctacctgatgcctgttcgcgcccgccgtgagctgcgcactggccgtggccaccattcacctctgtaatttaatccgtttctcttggattgtctggacgtgcccgatggttctttcttttgctcagtggagttggaggttttgtgtttggttttctcattcttgtcttgttttgtgtgggtggattttcaccgaccaatgatatcctcttctgacggtcaccttctttccacttcactggagtccagtattctgtaccacatcacgcaacgtgttatcttgtggtgtaaataaagactgcgttacttgccctcccaaaaa

SEQ ID NO: 2 - protein, 1084 aa, Homo sapiens
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDAERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 3 - DNA, 3960 bp, Mus musculus
cgggcctcctcaattttgttcctgcatgtgctggtggaaatttggagtgtgttggagccactgtttacctggacatcttgagccattttgaacttaaggcactgacgctgctagcaatgagctcccaaagccatccagatggactttctggccgagaccagcctgtggagctgctgaatcctgcccgtgtgaaccacatgcccagcacggtggacgtggctacagcgctgcctctgcaagtggcccctacagcagtacccatggacctgcgcttggaccaccagttctcactgcccttggaacctgcattgcgggagcagcaactgcagcaggaactcctagcactgaaacagaagcagcagatccagcggcagatactcattgcagagttccagcgtcaacatgagcagttgtcccgacagcatgaggcacagttgcatgaacatatcaagcagcagcaggagatgctggccatgaagcaccagcaggagctgctggagcaccagcggaaactggagcggcaccggcaagagcaggagctggagaagcagcaccgtgagcagaagctgcagcagctcaagaacaaggagaagggcaaagagagtgctgtggcgagcacagaggtgaagatgaagctgcaggagtttgttctcaacaagaagaaggctctagcccaccggaacctgaaccactgcatttccagcgatccccgctactggtatgggaagacacagcacagctcccttgaccagagctctccaccccagagtggggtgtcagcctcctacaaccaccccgtcttgggaatgtacgacgccaaagatgacttccctcttaggaaaacagcttctgaacctaacctgaaattacgctcaaggcttaagcagaaagtagctgagagacggagcagccccctgttgcgcaggaaagatggccctgtggccactgctctaaaaaagcgacccctggatgttacagactccgcatgcagcagcgcccctggctccggtcccagctctccaaatagcagctctggcaacgtcagcactgagaatggcatcgcacccactgtgcccagcgctccagctgagacgagcttggcacacagacttgtgactcgagaaggctcagtcgccccacttcctctctacacgtcaccatccttacccaacatcaccttgggacttcctgccactggccctgccgctggtgcggcaggtcagcaggatgctgagaggcttgctctcccagctctccagcagcggatcttgttccctgggacccacctcaccccgtacctgagcacctcgcccctggagagggacggtgcagcagctcacaaccccctcctgcagcacatggtcctgctggagcagccacccacccagacaccccttgtcacaggcctgggggcgctgcccctccactcacagtccctggttggtgcggacagggtgtccccatccattcacaagctgcggcagcaccgccctctggggcgcacgcagtcagcacccctgccgcagaacgcacaggccctgcagcacctggtgatccagcagcagcaccagcagttcctggagaagcacaagcaacagttccagcagcagcagctgcacctcagcaagataatctccaaacctagtgagccacctcggcagcctgagagccacccagaggagacagaggaggagctccgtgagcaccaggccttgctggatgagccctacctagatcggctacctgggcagaaggagccctccctggctggtgtgcaggtgaagcaggagcccattgagagtgaggaggaagaagcggaggccactcgagagacagagcccggccagcgcccagccactgagcaggagctgctcttcagacagcaagccctcctactggagcagcagaggatccaccagttaagaaactaccaggcatctatggaggctgctggcatccctgtgtcatttggcagccacagacctctgtctcgggcacagtcctccccagcatctgccaccttccccatgtcagtccaggagccccccaccaagccaaggttcaccacaggtcttgtgtatgacacactgatgttgaagcatcagtgcacctgtgggaacaccaacagccacccggagcatgctgggaggatccagagcatctggtcccgcctgcaggagactggactccgtggcaagtgtgagtgcatccgtggacgcaaggccacattggaggagctgcagacagtgcactcggaggcccacacactcctctacggcacaaatcctctcaacagacagaaactggacagctcgctgacctcagtgttcgtcaggcttccttgtggtggtgttggggtggatagcgacaccatatggaatgaggtgcactcgtctggggcagcccgcctggctgtaggctgtgtagtggagctggtcttcaaggtggccacgggagagctaaagaatggctttgctgtggttcgtcccccaggacaccatgccgaggagagcacacccatgggtttctgctactttaactccgtggcagttgcagccaaacttctccagcagaggctgaatgtgagcaagatcctcattgtagactgggatgtacatcatgggaatgggacccagcaggccttctacaatgaccccaatgttctctacatgtccctgcaccgctatgacgatgggaacttcttcccaggaagtggagcaccagatgaggtgggcacagggccaggcgtgggtttcaatgtcaacatggctttcacgggtggcctcgaaccccccatgggagacgctgagtacttggcagccttcagaacggtggttatgcctatcgcaaatgagtttgccccagatgtggtactggtgtcatcgggcttcgatgctgtggagggccaccccacacctcttggagggtacaatctctctgccaaatgttttgggtacttgacaaaacagctgatgggcttagctggtggccggcttgtgctggcccttgagggaggccatgacctgacagccatctgtgatgcttctgaagcctgcgtgtctgctctgctgggaaacgagcttgagcctctgccagaaaaggttctacatcagagacccaatgccaatgctgtccactccatggagaaagtgatggacatccacagcaagtactggcgctgcctgcagcgtctgtcctccacggtggggcactctctgattgaggcgcaaaagtgtgagaaggaagaagctgagacagtcaccgccatggcctcgctgtctgtaggcgtcaaacctgctgagaagagatctgaggaggagcccatggaggaggaaccaccactgtagcccgaagctgctgttctctcctttgtttgtctgtcttgaagctcagccaagaaacttacccgtatcactcctgcgtcctgccttggtgctctcttgagtacaaagggacagcaggcgtgcagcagcaacgggaagcctttctgccgccctggaccacaggtctggagacgcacacggaacaccgggtgtggcagatcacatggaacacaggacagcgcgcaacacacgggcacacagacatgtggaagccaagcacacgctggtggctccccctccccctgggaagcctcgaaggaagaagcttgtggcaacttgggcagagttgctgaatcaagttgacatgaggaaaatgaaaatcaaagatctaataatacagaacaaacttgattaaaactggtgcttaacatttgattcttacccacaatcccacagtctccgtgtaaaccactcaactcatcttgtagcttttttttttttttaaacgaaaacaaaaccattttctatggctctggcctgccttgtgaaccttagtggggtgctgtgggggttcacagcttagtgagggacagagttggaagaaagtttaaacgaaaaaaaaaaaaaaaaaaaaaaa

SEQ ID NO: 4 - protein, 1076 aa, Mus musculus
MSSQSHPDGLSGRDQPVELLNPARVNHMPSTVDVATALPLQVAPTAVPMDLRLDHQFSLPLEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSASYNHPVLGMYDAKDDFPLRKTASEPNLKLRSRLKQKVAERRSSPLLRRKDGPVATALKKRPLDVTDSACSSAPGSGPSSPNSSSGNVSTENGIAPTVPSAPAETSLAHRLVTREGSVAPLPLYTSPSLPNITLGLPATGPAAGAAGQQDAERLALPALQQRILFPGTHLTPYLSTSPLERDGAAAHNPLLQHMVLLEQPPTQTPLVTGLGALPLHSQSLVGADRVSPSIHKLRQHRPLGRTQSAPLPQNAQALQHLVIQQQHQQFLEKHKQQFQQQQLHLSKIISKPSEPPRQPESHPEETEEELREHQALLDEPYLDRLPGQKEPSLAGVQVKQEPIESEEEEAEATRETEPGQRPATEQELLFRQQALLLEQQRIHQLRNYQASMEAAGIPVSFGSHRPLSRAQSSPASATFPMSVQEPPTKPRFTTGLVYDTLMLKHQCTCGNTNSHPEHAGRIQSIWSRLQETGLRGKCECIRGRKATLEELQTVHSEAHTLLYGTNPLNRQKLDSSLTSVFVRLPCGGVGVDSDTIWNEVHSSGAARLAVGCVVELVFKVATGELKNGFAVVRPPGHHAEESTPMGFCYFNSVAVAAKLLQQRLNVSKILIVDWDVHHGNGTQQAFYNDPNVLYMSLHRYDDGNFFPGSGAPDEVGTGPGVGFNVNMAFTGGLEPPMGDAEYLAAFRTVVMPIANEFAPDVVLVSSGFDAVEGHPTPLGGYNLSAKCFGYLTKQLMGLAGGRLVLALEGGHDLTAICDASEACVSALLGNELEPLPEKVLHQRPNANAVHSMEKVMDIHSKYWRCLQRLSSTVGHSLIEAQKCEKEEAETVTAMASLSVGVKPAEKRSEEEPMEEEPPL

SEQ ID NO: 5 - DNA, 3318 bp, Canis lupus
  Canis lupus familiaris
atggtgggctctccctactgtgggcccgtagcgggagcgcagagtgaccgccacgtcagcgtctataatgacgtgcctgtcactgcagatggactttctggccgagaccagcccgtggagctgctaaaccctgcccgcgtgaaccacatgcccagcacggtggatgtggcctccgcgctgcctctgcaggtcgcccccccggcggtgcccatggacctccgcctggaccaccagtttgcgctgcccgtggcggagcccaccctccgggagcagcagctgcagcaggagctactggccctcaagcagaagcagcagctccagaggcagatcctcatcgccgagttccagaggcagcacgagcagctctcccggcagcacgaggcccagctgcacgagcacataaaacaacagcaggagctgctggccatgaagcatcagcaggagctgctcgagcaccagcggaagctggagaggcatcgccaggagcaggagctggagaagcagcaccgggagcagaagcttcagcagctcaagaacaaggagaaaggcaaagagagtgcggtggccagcacggaagtgaagatgaagctgcaggagtttgtcctcaataaaaagaaggcgctggcccaccggaacctgaaccactgcatctccagcgacccccgctactggtacgggaaaacacagcacagttcgctcgaccagagctccccaccccagagcggggtgtcggcctcctacaaccacccggtcctgggaatgtacgacaccaaagatgacttcccgctcagaaaaacagcttctgaacccaatttgaaactacggtccaggctaaagcagaaggtggccgagagacggagcagccccctgctacgcagaaaagatgggccagtggtcaccgctctgaaaaagcgtccgctggacgtcacagactcggcgtgcagcagtgcccccggctccgctcccagctcccccaacaacagctccgggaacgtcagcgcggagaacgggatcacacctgccgtggcgagcatcccggccgagaccggcttggcgcacagactcgtgactcgagaaggctccgtggccccgctccccctgtacacgtcaccgtccctgcccaacatcacgctgggactgcccgccaccggcccttccgcgggggcggccggccagcaggacgccgagcgactcgctctcccggccctccagcagcggatccccctcttccccggcacccacctcactccctacctgagcacggcgcccctggagcgggacggcggggctgcgcacaaccccctcctgcagcacatggtcttactggagcagacgcctgcacagacgcccctcgtcacaggcctgggcgcgctgcccctgcacgcgcagcctctggtgggtgcggaccgcgtgtccccgtccatgcacaagctgcggcagcaccgcccgctggggcgcacccagtcggcgccgctgccccagaacgcgccggccctgcagcacctggtgatccagcagcagcaccagcagttcctggagaagcacaagcagcacttccagcagcagctgcacatgaacaagattatccccaagcagaacgagccagcccggcagcccgagagccaccccgaggagacggaggaggagcttcgcgagcaccaggccctcctggaggagccctacctggaccggctgtcggggcagaaggaggcgcacgccctggccggggtgcaggtgaagcaggagcccatcgagagcgacgacgaggaggcggagcccccgcgggaggtggagccgggccagcgccagcccacggagcaggagctgctcttcagacagcaagccctccttctggaacagcagcggatccaccagctgagaaactaccaggcgtccatggaggccgctggcatccccgtgtcctttggcggccaccggcctctgtcgcgggcgcagtcctccccggcgtctgccaccttccccatgtctgtgcaggagccgcccaccaaaccaaggttcaccacaggcctcgtgtacgacaccctgatgctgaagcaccagtgtacctgcgggaacaccaacagccaccccgagcacgccggcaggatccagagcatctggtctcggctgcaggagacggggctccggggcaagtgtgagtgcatccgcggacggaaggccactctggaggagctgcagaccgtgcattcggaggcgcacaccctgctctacggcacaaaccccctcaacaggcagaaactggacagtaagaaacttctaggctcgctaacgtcggtgttcgtcaggctgccctgcggtggtgtcggggtggacagcgacaccatatggaacgaggtgcactcgtcgggggcggcgcgcctggccgtgggctgcgtggtggagctggtcttcaaggtggccaccggggagctgaagaacggctttgccgtggtccgccctccaggacatcacgcggaggagagcacgcccatgggcttctgctactttaattctgtggccatcgcagccaagcttctccagcagcggctggacgtgagcaagaccctcatagtggactgggacgtgcaccacggaaatgggacccagcaggccttctacaacgacccgaacgtcctgtacatctcccttcaccgctacgacgacgggaacttcttcccgggcagcggggccccggacgaggtgggcacagggccaggtgtgggcttcaacgtcaacatggctttcactggtggccttgacccccccatgggagacgcggagtacttggcagccttcaggaccgtggtcatgcccatcgcgaacgagtttgccccagacgtggtgctcgtgtcatcaggctttgacgctgtggagggccaccccacgcccctcggcggctacaacctctccgccaaatgtttcgggtacctgacgaagcagctgatgggcttggctggcggccggatcgttctggccctggaggggggccacgacctcacggccatctgcgacgcctcggaagcatgtgtttctgctttgctgggcaatgagcttgatcctctcccagaaaaggttttacagcagagacccaacgctaacgctgtccggtccatggagaaagtcattgagatccacagtgagttgtgtgggccacgtgctcggcggggagccccagggcactctctgttagacgcccagaagtgcgagaacgaggaagccgcagactgtcatccccctattgtctccctgtttgagggcgtcaagaccgcggagaaaagacccgacgaggagcccatggaagaggagccgcccctgtag

SEQ ID NO: 6 - protein, 1105 aa, Canis lupus
  Canis lupus familiaris
MVGSPYCGPVAGAQSDRHVSVYNDVPVTADGLSGRDQPVELLNPARVNHMPSTVDVASALPLQVAPPAVPMDLRLDHQFALPVAEPTLREQQLQQELLALKQKQQLQRQILIAEFQRQHEQLSRQHEAQLHEHIKQQQELLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSASYNHPVLGMYDTKDDFPLRKTASEPNLKLRSRLKQKVAERRSSPLLRRKDGPVVTALKKRPLDVTDSACSSAPGSAPSSPNNSSGNVSAENGITPAVASIPAETGLAHRLVTREGSVAPLPLYTSPSLPNITLGLPATGPSAGAAGQQDAERLALPALQQRIPLFPGTHLTPYLSTAPLERDGGAAHNPLLQHMVLLEQTPAQTPLVTGLGALPLHAQPLVGADRVSPSMHKLRQHRPLGRTQSAPLPQNAPALQHLVIQQQHQQFLEKHKQHFQQQLHMNKIIPKQNEPARQPESHPEETEEELREHQALLEEPYLDRLSGQKEAHALAGVQVKQEPIESDDEEAEPPREVEPGQRQPTEQELLFRQQALLLEQQRIHQLRNYQASMEAAGIPVSFGGHRPLSRAQSSPASATFPMSVQEPPTKPRFTTGLVYDTLMLKHQCTCGNTNSHPEHAGRIQSIWSRLQETGLRGKCECIRGRKATLEELQTVHSEAHTLLYGTNPLNRQKLDSKKLLGSLTSVFVRLPCGGVGVDSDTIWNEVHSSGAARLAVGCVVELVFKVATGELKNGFAVVRPPGHHAEESTPMGFCYFNSVAIAAKLLQQRLDVSKTLIVDWDVHHGNGTQQAFYNDPNVLYISLHRYDDGNFFPGSGAPDEVGTGPGVGFNVNMAFTGGLDPPMGDAEYLAAFRTVVMPIANEFAPDVVLVSSGFDAVEGHPTPLGGYNLSAKCFGYLTKQLMGLAGGRIVLALEGGHDLTAICDASEACVSALLGNELDPLPEKVLQQRPNANAVRSMEKVIEIHSELCGPRARRGAPGHSLLDAQKCENEEAADCHPPIVSLFEGVKTAEKRPDEEPMEEEPPL

SEQ ID NO: 7 - DNA, 4015 bp, Rattus norvegicus
ggagcgcctgacgggcgtagggcgcggcctatccccgcgcggctctgcgctcgagggggagcagcatcatggttcaacgttcatctctgcaaggccaggtgacaacttctgctcttcttgtggattacttggtttacaagacgctgccagtgggacttcgagtttgcgcagctgggacccgggcctcctcagttttgtacctgcatgttctcgtgggaatttggagcgtgttggagccactgtttacctggacatcttgagccatttcgaacttaaggcactgacgctgctagcaatgagctcccaaagccatccagatggactttctggccgagaccagcctgtggagctgctgaatcctgcccgcgtgaaccacatgcccagcacggtggacgtggctacagcgctgcctctgcaggtggcccctgcagcagtacccatggacctgcgcttggaccaccagttctcactgcccttggaacctgcactgcgggagcagcaactgcagcaggaactcctagcactgaagcagaagcagcagatccagcggcagatcctcattgcagagttccagcgtcagcatgagcagttgtcccgacagcatgaggcacagctgcatgaacatatcaagcagcagcaggagatgctggccatgaagcaccagcaggagctgctggagcaccagcggaaactggagcggcaccggcaagagcaggagctggagaagcagcaccgtgagcagaagctgcagcagctcaagaacaaggagaagggcaaagagagtgctgtggcgagcacagaggtgaagatgaagctgcaggagtttgttctcaacaagaagaaggctctggcccaccggaacctgaaccactgcatgtccagcgacccccgctactggtatgggaagacacagcacagctcccttgaccagagctctccaccccagagtggggtgtcagcctcctacaaccaccctgtcttgggaatgtacgacgccaaagatgacttccctcttaggaaaacagcttctgaacctaacctgaaattacggtcaaggcttaagcagaaagtagctgagagacggagcagccccctgctgcgcaggaaagatggccctgtggccactgctctaaaaaagcgacccctggatgttacagactccgcatgtagcagcgcccctggctccggtcccagctcccccaatagcagctctggcaacgtcagcaccgagaatggcattgcacccaccgtgcccagcactccagctgagacgagcttggcacacagacttgtgactcgagaaggctctgttgccccacttcctctctatacatcaccatccttgcccaacatcaccctgggacttcctgccactggccctgctgctggtgcagcaggccagcaggatgctgagaggcttgctctcccagccctccagcagcgaatctccttgttccctgggacccacctcaccccatacctgagcacctcgcccctggagcgggacggtggagcagctcacaaccccctcctgcagcacatggtcctgctggagcagccacccacccagacgccccttgtcacaggcctgggggcactgcccctccacacacagtccctggttggtgcggacagggtgtccccatccattcacaagctgcggcagcaccgacctctggggcgcacgcagtcagcacccctgccgcagaacgcacaggccctgcagcacctggtgatccagcagcagcaccagcagtttctggagaagcacaagcaacagttccagcagcagcagctgcacctcagcaagatgatttctaaacccagtgagccacctcggcagcctgagagccacccggaggagacagaggaggagctccgtgaacaccaggccttgctggatgagccctacctagatcggctccctgggcagaaggagccctccttagctggtgtgcaggtgaagcaggagcccatcgagagtgaggaggaagaagtagaggccactcgagaggcggaacccagccagcgcccagccactgaacaggagcttctctttagacagcaagctcttctgctggagcagcagaggatccaccagttaaggaactaccaggcatctatggaggctgctggcatccctgtgtcatttggcagccacagacctctgtctcgcgcacagtcctccccagcatctgccaccttccccatgtcagtccaggagccccccaccaaaccaaggttcaccacaggccttgtgtatgacacactcatgttgaagcaccagtgcacctgtgggaacaccaacagccacccggagcatgctgggaggatccagagcatctggtcccgcctgcaggagactggcctccgtggcaagtgtgagtgcatccgcggacgcaaggccacactggaagagctgcagacagtgcactcggaggcccacacactcctctatggcacaaaccctctcaacagacagaaactggacagctcgctgacctccgtgtttgtcaggcttccttgtggtggtgttggggtggatagtgataccatatggaatgaggtgcactcgtctggggcagcccgcctggctgtaggctgtgtagtggagctggtcttcaaggtggccacgggagagctaaagaatggctttgctgtggttcgtcccccaggacaccatgccgaggagagtacacccatgggtttctgctactttaattctgtggcaattgcagccaaacttctccagcagaggttgaatgtgagcaagatcctcattgtagactgggatgtgcaccatgggaatgggacccagcaggccttctacaatgaccccaatgttctctacatgtccctgcaccgctatgacgatgggaacttcttcccaggaagtggagcaccagatgaggtgggcacagggccaggcgtgggtttcaatgtcaacatggccttcacgggtggcctcgatccccccatgggagacgccgagtacctggcagccttcagaacggtggtcatgccaatcgcaaatgagtttgccccagatgtggtactggtgtcctcgggcttcgatgctgtggagggccaccccacacctcttggagggtacaatctctctgccaaatgtttcgggtacttgacaaaacagctgatgggcttagctggtggccggatcgtgctggccctggagggaggccatgacctgacagccatctgtgatgcttctgaagcatgtgtttctgctctgctgggaaatgagcttgagcctctgccagaaaaggttctacatcagagacccaatgccaatgccgtccactccatggagaaagtgatgggcatccacagtgagtactggcgctgcctgcagcgtttgtcccccactgtggggcactctctgattgaggcgcagaagtgtgagaatgaagaagctgagacagtcaccgccatggcctcgctgtctgtaggcgtcaaacctgctgagaagagatctgaggaggagcccatggaggaggagccaccactgtagccgaagctgctgttctctcctttgtctgtctgtcttaaggctcagccaagaaactttcccgtgtcactcctgcgtcctgccttggtgctctctctgagtgcgaagggacagcaggtgtgaagcagcaacgggaggcctttctgccgccctggaccacaggtctggagacgcacacgaacaccaggtgtggcagatcagatggaacacaggacagtgcgcaacacgtgggcacacagacatgccgcagaagccaagcacacgctgctggggtcccccaggaaagccacgaaggaagaagcttgtggcaacttgggcagagttgctgaattgagttgacatgaggaaaatgaaaatcaaagatctaataatacagaacaaacttgattaaaactggtgcttaacatttgattcttacccacaatcccacagtctccgtgtaaaccactcaactcatcttgtagcttttttaaaaaaaaaaaaaaacaa

SEQ ID NO: 8 - protein, 1077 aa, Rattus norvegicus
MSSQSHPDGLSGRDQPVELLNPARVNHMPSTVDVATALPLQVAPAAVPMDLRLDHQFSLPLEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCMSSDPRYWYGKTQHSSLDQSSPPQSGVSASYNHPVLGMYDAKDDFPLRKTASEPNLKLRSRLKQKVAERRSSPLLRRKDGPVATALKKRPLDVTDSACSSAPGSGPSSPNSSSGNVSTENGIAPTVPSTPAETSLAHRLVTREGSVAPLPLYTSPSLPNITLGLPATGPAAGAAGQQDAERLALPALQQRISLFPGTHLTPYLSTSPLERDGGAAHNPLLQHMVLLEQPPTQTPLVTGLGALPLHTQSLVGADRVSPSIHKLRQHRPLGRTQSAPLPQNAQALQHLVIQQQHQQFLEKHKQQFQQQQLHLSKMISKPSEPPRQPESHPEETEEELREHQALLDEPYLDRLPGQKEPSLAGVQVKQEPIESEEEEVEATREAEPSQRPATEQELLFRQQALLLEQQRIHQLRNYQASMEAAGIPVSFGSHRPLSRAQSSPASATFPMSVQEPPTKPRFTTGLVYDTLMLKHQCTCGNTNSHPEHAGRIQSIWSRLQETGLRGKCECIRGRKATLEELQTVHSEAHTLLYGTNPLNRQKLDSSLTSVFVRLPCGGVGVDSDTIWNEVHSSGAARLAVGCVVELVFKVATGELKNGFAVVRPPGHHAEESTPMGFCYFNSVAIAAKLLQQRLNVSKILIVDWDVHHGNGTQQAFYNDPNVLYMSLHRYDDGNFFPGSGAPDEVGTGPGVGFNVNMAFTGGLDPPMGDAEYLAAFRTVVMPIANEFAPDVVLVSSGFDAVEGHPTPLGGYNLSAKCFGYLTKQLMGLAGGRIVLALEGGHDLTAICDASEACVSALLGNELEPLPEKVLHQRPNANAVHSMEKVMGIHSEYWRCLQRLSPTVGHSLIEAQKCENEEAETVTAMASLSVGVKPAEKRSEEEPMEEEPPL

SEQ ID NO: 9 - DNA, 9016 bp, Pan troglodytes
agcatcatggttcaacgtgggtcaattttttgtgaatgaggaggggaggttgtggggccgccgccgcggagcaccgtccccgccgccgcccgagcccgagcccgagcccgcgcacccgcccgcgccgccgccgccgccgcccgaacagcctcccagcctgggcccccggcggcgccgtggccgcgtcccggctgtcgccgcccgagcccgagcccgcgcgccggcgggtggcggcgcaggctgaggagatgcggcgcggagcgccggagcagggctagagccggccgccgccgcccgccgcggtaagcgcagccccggcccggcgcccgcgggccattgtccgccgcccgccccgcgccccgcgcagcctgcaggccttggagcccgcggcaggtggacgccgccggtccacacccgccccgcgcgcggccgtgggaggcgggggccagcgctggccgcgcgccgtgggacccgccggtccccagggccgcccggccccttctggacctttccacccgcgccgcgaggcggcttcgcccgccggggcgggggcgcgggggtgggcacggcaggcagcggcgccgtctcccggtgcggggcccgcgccccccgagcaggttcatctgcagaagccagcggacgcctctgttctccttgtgggttacctggctcatgagaccttgccggcgaggctcggcgcttgaacgtctgtgacccagccctcaccgtcccggtacttgtatgtgttggtgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcctgcccgcgtgaaccacatgcccagcacggtggatgtggccgcggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgccgagttccagaggcagcacgagcagctctcccggcagcatgaggcgcagctccacgagcacatcaagcaacagcaggagatgctggccatgaagcaccagcaggagctgctggaacaccagcggaagctggagaggcaccgccaggagcaagagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccaccgtccccagcatcccggcggagacgagtttggcgcacagacttgtggcacgagaaggctcggccgctccacttcccctctacacgtcgccatccctgcccaacatcacgctgggcctgcctgccaccggcccctctgctggcacggcgggccagcaggacgccgagagactcgcccttcccgccctccagcagaggctctcccttttccccggcacccacctcactccctacctgagcacctcgcccttggagcgggacggaggggcagcgcacagccctcttctgcagcacatggtcttactggagcagccgccggcacaagcgcccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgacgaagaggcagagcccccacgggaggtggagccaggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagcagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccctgcggtggtgttggggtggacagtgacaccatatggaacgaggtgcactcggcgggggcagcccgcctggctgtgggctgcgtggtagagctggtcttcaaggtggccacgggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccccagcgtcctgtacgtgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgacgccgtggagggccaccccacccctcttgggggctacaacctctccgccagatgcttcgggtacctgacgaagcagctgatgggcctggctggcggccggatcgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacgggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaagaagcctgtggcaacaggcggccgagctgccgaattcagttgacacgaggcacagaaaacaaatatcaaagatctaataatacaaaacaaacttgattaaaactggtgcttaaagtttattacccacaactccacagtctctgtgtaaaccactcgactcatcttgtagctttttttttttttaaaaaaggacgttttctacggctgtggcccgcctctgtgaaccacggcggtgtgcggcggggggtctgcacccgggtgggggacagagggacctttaaagaaaacaaaactggacagaaacaggaatgtgagctgggggagctggcttgagtttctcaaaagccatcggaagatgcgagtttgtgccttttttttattgctctggtggatttttgtggctgggttttctgaagtctgaggaacaatgccttaagaaaaaacaaacagcaggaatcggtgggacagtttcctgtggccagccgagcctggcagtgctggcaccgcgagctggcctgacgcctcaagcacgggcaccagccgtcatctccggggccaggggctgcagcctggcggtccctgttttgctttattgctgtttaagaaaaatggaggtagttccaaaaaagtggcaaatcctgttggaggttttgaagtccaacaaattttaaacgaatccaaagtgttctcacacgtcacatacgattgagcatctccatctggtcgtgaagcatgtggtaggcacacttgcagtgttacgatcggaatgctttttattaaaagcaagtagcatgaagtattgcttaaattttaggtataaataaatatatatatgtataatatatattccaatgtattccaagctaagaaacttgattcttatgaaatcttgataaaatatttataatgcatttatagaaaaagtatatatatatataaaatgaatgcagattgcgaaggtccctgcaaatggatggcttgtgaatttgctctcaaggtgcttatggaaagggatcctgattgaaattcatgttttctcaagctccagattggctagatttcagatcgccaacacattcgccactgggcaactaccctacaagtttgtactttcattttaattattttctaacagaacccctcccgtctccaagccttcatgcacatatgtacctaatgagtttttatagcaaagagtataaatttgctgttgatttttgtatgaattttttcacaaaaagatcctgaataagcattgttttatgaatcttacatttttcctcaccatttagcaattttctgaatggtaataatgtctaaatctttttcctttctgaattcttgcttgtacatttttttttttacctttcaaaggtttttaattatttttgtttttatttttgtacgatgagttttctgcagcgtacagaattgttgctgtcagattctattttcagaaagtgagaggagggaccgtaggtcttttcggagtgacaccaacgattgtgtctttcctggtctgtcctaggagctgtataaagaagcccaggggctctttttaactttcaacactagtagtattacgaggggtggtgtatttttcccctccgtggcaagggcagggagggttgcttaggatgcccggccaccctgggaggcttgccagatgccgggggcagtcagcattaatgaaactcatgtttaaacttctctgaccacatcgtcaggatagaattctaacttcagttttccaaagaccttttgagcatgtcagcaatgcatggggcacacgtggggctctttacccacttgggtttttccactgcagccacgtggccagccctggattttggagcctgtggctgcaaggaacccagggacccttgttgcctggtgaacctgcagggagggtatgatttcctgaccaggacagccagtctttactctttttctcttcaacagtaactgacagtcacgttttactggtaacttattttccagcacatgaagccaccagtttcattccaaagtgtctattgggttcagacttgtgggcagaagttcagacacaccgtgctcaggagggacccagagccgagtttcggagtttggtaaagtttacagggtagcttctgaagttaactcaaacttttgaccaaatgagtgcagattcttggattcacttggtcattgggctgctgatggtcagctctgagacagtggtttgagagcaggcagaacggtcttgggacttgtttgactttcccctccctagtggccactctttgctctgaagcccagattggcaagaggagctggtccattccccattcatggcacagagcagcggcagggcccagctagctggctcttctggcctccttggcctcattctctgcacagccctctagggatcctgccacctgccctcttaccccgccgtggcttatggggaggaatgcatcatctcacttttttttttaaagcagatgatgggataacatggactgctcagtggccaggttatcagtggggggacttaattctaatctcgttcaaatggagacaccctctgcaaaggcctggcagggggaggcacgtttcatctgtcagctcactccagcttcacaaatgtgctgagagcattactgtgtagccttttctttgaagacacactcggctcttctccacagcaagcgtccagggcagatggcggaggatctgcctcggcgtctgcaggcgggaccacgtcagggagggttccttcatgtgttctccctgtgggtccttggacctttagcctttttcttcctttgcaaaggccttgggggcactggctgggagtcagcaagcgagcactttatatccctttgagggaaaccctgatgacgccactgggcctcttggcgtctgccctgccctggcggcttcccgccgtgccgcagcgtgcccacgtgcccacgccccaccagcaggcggctgtcccggaggccgtggcccgctgggactggccgcccctccccagcgtcccagggctccggttctggagggccactttgtcaaggtgtttcagtttttctttacttcttttgaaaatctgtttgcaaggggaaggaccatttcgtaatggtctgacacaaaagcaagtttgatttttgcagcactagcaatggactttgttgtttttctttttgatcagaacattccttctttactggtcacagccacgtgctcattccattcttctttttgtagactttgggcccacgtgttttatgggcattgatacatatataaatatatagatataaatatatatgaatatatttttttaagtttcctacacctggaggttgcatggactgtacgaccggcatgactttatattgtatacagattttgcacgccaaactcggcagctttggggaagaagaaaaatgcctttctgttcccctctcatgacatttgcagatacaaaagatggaaatttttctgtaaaacaaaaccttgaaggagaggaggggggggggacgtttgcgtcttattgaacttattcttaagaaattgtactttttattgtaagaaaaataaaaaggacttaaacatttgtcatattaagaaaaaaagtttatctagcacttgtgacataccaataatagagtttattgtatttatgtggaaacagtgttttagggaaactactcacaattcacagtgaactgcctgtctctcttgagttgatttggacgaattttgttttgttttgttttgtgtttccttttatctccttccacgggccaggcgagcgtcgcccgccctcactggccttgtgacggtttattctgattgagaactgggcggactcgaaagagtgtccccttttccgcacagctgtgttgactttttaattacttttaggtgatgtatggctaagatttcactttaaagcagtcgtgaactgtgcgagcactgtggtttaaaattatactttgcatcgaaaggaaaccatttcttcattgtaacgaagctgagcgtgttcttagctcggcctcactttgtctctggcattgattaaaagtctcctattgaaagaaaaagaaagcgaacagtttttgtttggtttttttgccgtgtgtgctccatagtggaggcgctattttccaattgatgagaatgacaaacatatataatctatctatctatctatcatctatctatctatctataaagggggcttgaaccttactcacccaagagcttcttacggaatgtggtagaaaaccaagttgtaacaacactgtaacctgcctgatgcctgttcgcgcccggcgtgagctgcgcactggccgtggccaccattcacctctgtaatttaatccgtttctcttggattgtctggacgtgcccgatggttctttcttttgctcagtggagttggaggttttgtgtttggttttctcattctcgtcttgttttgtgtgggtggattttcaccgaccaatgatatcctcttctgacggtcaccttctttccacttcactggagtccagtattctgtaccacatcacgcaacgtgttatcttgtggtgtaaataaagactgcgttacttgccctccca

SEQ ID NO: 10 - protein, 1084 aa, Pan troglodytes
MSSQSHPDGLSGRDQPVELLNPARVNHMPSTVDVAAALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPTVPSIPAETSLAHRLVAREGSAAPLPLYTSPSLPNITLGLPATGPSAGTAGQQDAERLALPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDDEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYVSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 11 - DNA, 4554 bp, Sus scrofa
atgaagctggcgtcgatgctgctaaaagtaacccagttattacagggggacgcatctgtgactctgccctcgggtatcctgtgtggggacactccgggggagcatcctgaccgccaggttcccctcggtgggggtgggtggagcgcgtttggtcccggggtggtgggcgctggctgggaggccggcctcagggctgggtttggccgggctctgctttgcagcgtggtttcccgacgccggctgggtggtgcagagccggcctcgccgagcctttggggctgccgtccggccctgagcctcgcggctgcccagctgcaggcgctcgtccgggaaagcgacccccatgctgccgaggccccagcccgaggaccgccaccccagggtgacctgcaggccccggctccccgctgtgcctccagagccctgcacgaagccgcacgcacagaaatcacaacccagaggacgagagcgcaggtggagagggacgtggagctagaccagcaggctcgcaaccctgagcccgggaccgggaccgccgctgtgaccccacccccgggcctccggacacccccacccccgctcgcagggggtgctggtgcatccacaccccaggccactcagcacgggacaggaggggcttccctgcagggctcctcgctgccccgcgcccatccatgctggcatctgtgggatgacaagccgctggggctccctgtgacgccggagaacctgagccaagctggagggaagaaagctggcatgtcccttccagcccagtcctgcctggccgcctgcagcaccctgccctctgcctctctgtggccgcctcccatctccagcagggccctccaggacctgcaggtgcaggctctgccctcggagcgggggacccggacctgccacaggcccgactgggagcagtccccgtgccgggatggagcaggagccgtgcccgtgtctgcggtgtttgcatgcccgcctctggctcggcagggcgttcgtgccgcgcagcagacctgctgctttgccaagcgcggaaacaccgccccatgccagctcgtgccggccagcgcgccgggagcacctctcattggctgccgccgggaagcacgtgttccagagggcgcgcgcgtcaccatggcaacgcgctcgaggccgagcgccgcctggaccgccgagccccgcagtctgttccggatgacgtccgaagaggccgctgggccggcagcgaggtcctttgggcgagcagggccccctctcgtggacgtggatgtggcctcggcgctgcctctgcaagtggctcccccggcggtgcccatggacctgcgcctggaccaccagttcccgctgcccgtgacggagcccggcctccgggagcagcagctgcagcaggagctcctggccctgaagcagaagcagcagctgcagaggcagatcctcattgccgagttccagaggcagcacgagcagctgtcccggcagcatgaagcccagctccacgagcacatcaaacaacagcaagagctgctggccatgaagcaccagcaggagctgttggagcatcagcggaagctggagaggcaccgccaggagcaggagctggagaagcagcaccgggagcagaagctgcagcagctcaagaacaaggagaaaggcaaagagagtgccgtggccagcacggaagtgaagatgaagctacaagagtttgtcctcaacaaaaagaaggcgctggcccaccggaacctgaaccactgcatgtccagcgacccccgctactggtacgggaagacacagcacagctccctggaccagagctccccgccccagagcggggtgtccgcctcctacaaccacccggtcctgggcatgtatgacgccaaagatgactttccgctcagaaagacagcttctgaacccaatctgaaattacgctccaggctaaagcagaaagtggccgagagacggagcagccctctgttacgcaggaaagatgggcctgtggttactgctctaaaaaagcgtcccctggatgtcacagactccgcgtgcagcagcgccccgggctccgcgcccagctcgcccaacaacagctccgggagcgtcagcacggagaacggcatcgcgcccgctgtgcccagcgtcccggccgagaccagcttggctcacaggctcgtgactcgggaaggctccgtgggccccctccccctctacacgtccccgtctttgcccaacatcacgctggggcttcccgccaccggcccttccacggcggcgggccagcaggaagccgagaggctcaccctccccgccctccagcagcgcatccacctcttccccggcacccacctctccccctacctgagcgcggctcccctggaccgggacgcggggacggcccacaaccctctcctgcagcacatggtcctcctggagcagccggccgcgcagacacccctggtcacaggcctgggagcgctgcccctgcacgcgcagtccctggtgggggcggaccgcgtgtccccgtccatccacaagctgcggcagcaccgcccgctggggcgcacgcagtcggcgccactgccccagaacgcgcaggccctgcagcacctggtgatccagcagcagcaccagcagttcctggagaagcacaagcagcagtttcagcagcagcagctgcacctcaacaagattctccccaagcccagcgagccggcccggcagcccgagagccaccctgaggagacagaggaggagctgcgggagcaccaggccctcctggaggagcccttcctggaccggctctcagggcagaaggaggtgcacgcactgggggtgcaggtgaagcaggagcccgtcgagagtgacgacgaggaggccgagcccccgcgggaggcggaaccaggccagcggcagcccagggagcaggagctgctcttcagacaggtcctcgcctaccttatcctgcggacagaagagacagtgtttgatcagatgccaggacctttaggggacaagaaggcgccagtgacagccccacagatgcggaagagcagcatgcccaggtccccggggtcggcaagcgccctcctggagcagcagcgcattcaccagctgaggaactaccaggcgtccatggaggccgccggcgtccccgtgtccttcggcggccaccggcctctgtcccgggcacagtcctcccccgcgtccgccaccttccccatgtctgtgcaggagccccccgccaaaccaaggttcaccacaggtctcgtgtacgacacgctgatgctgaagcaccagtgcgcctgcgggaacaccaacagccacccggagcatgctgggaggatccagagcatctggtccaggctgcaggagactggccttcgaggcaaatgtgagtgcatccgcggacgcaaggccaccctggaggagctgcagacagtgcattcggaggcccacgccctgctctacggcacaaaccccctcaacaggcagaaactggacagtaagaaacttctaggctcgctcacgtcggtgtttgtcaggctgccctgcggtggtgtcggcgtagacagcgacaccatatggagcgaggtgcactcgtcgggggccgcccggctggccgtgggctgcgtggtggagctggccttcaaggtggccacaggggagctgaagaacggctttgctgtggtccgccctccgggacaccacgcggaggagagcacgcccatggggttctgctacttcaactccgtggccatcgccgccaagcttctccagcaaaggctgcacgtgagcaagaccctcgtagtggactgggacgtgcaccacgggaacgggacccagcaggccttctacagcgaccccagcgtgctctatgtctcgctgcaccgctacgacgacggcaacttcttcccgggcagcggggctcctgacgaggtgggcacagggccaggcgtgggcttcaacgtcaacatggctttcacgggcggcctggacccccccatgggagacgccgagtacttggcggccttcagaactgtggtcatgccgatcgccaacgagtttgccccagacgtggtgctggtgtcatcaggcttcgacgccgtggagggccaccccacgcctctcggcggctacaacctctctgccaaatgttttgggtacctgacgaagcagctgatgggcttggccggcggccgggtggtcctggccctggaggggggtcacgacctcacagccatctgtgacgcctcggaggcctgcgtctctgctttgctgggaaacgagcttgaccctctcccagaaaaggttttgcagcagagaccctacgacaactccgtgcggtccatggagaaggtcattgacatccacagccagtactggcgctccctgcagcgtctcgcctccaccgtgggctactccctggtcgaggcccagaagtgcgagaacgaggaagccgagacggtcactgccatggcctcgctgtctgtgggcgtcaaggcacccgagaagagacccgaggaggagcccatggaagaggagccgcccctgtag

SEQ ID NO: 12 - protein, 1517 aa, Sus scrofa
MKLASMLLKVTQLLQGDASVTLPSGILCGDTPGEHPDRQVPLGGGGWSAFGPGVVGAGWEAGLRAGFGRALLCSVVSRRRLGGAEPASPSLWGCRPALSLAAAQLQALVRESDPHAAEAPARGPPPQGDLQAPAPRCASRALHEAARTEITTQRTRAQVERDVELDQQARNPEPGTGTAAVTPPPGLRTPPPPLAGGAGASTPQATQHGTGGASLQGSSLPRAHPCWHLWDDKPLGLPVTPENLSQAGGKKAGMSLPAQSCLAACSTLPSASLWPPPISSRALQDLQVQALPSERGTRTCHRPDWEQSPCRDGAGAVPVSAVFACPPLARQGVRAAQQTCCFAKRGNTAPCQLVPASAPGAPLIGCRREARVPEGARVTMATRSRPSAAWTAEPRSLFRMTSEEAAGPAARSFGRAGPPLVDVDVASALPLQVAPPAVPMDLRLDHQFPLPVTEPGLREQQLQQELLALKQKQQLQRQILIAEFQRQHEQLSRQHEAQLHEHIKQQQELLAMKHQQELLEHQRKLERHRQEQELEKQHREQKLQQLKNKEKGKESAVASTEVKMKLQEFVLNKKKALAHRNLNHCMSSDPRYWYGKTQHSSLDQSSPPQSGVSASYNHPVLGMYDAKDDFPLRKTASEPNLKLRSRLKQKVAERRSSPLLRRKDGPVVTALKKRPLDVTDSACSSAPGSAPSSPNNSSGSVSTENGIAPAVPSVPAETSLAHRLVTREGSVGPLPLYTSPSLPNITLGLPATGPSTAAGQQEAERLTLPALQQRIHLFPGTHLSPYLSAAPLDRDAGTAHNPLLQHMVLLEQPAAQTPLVTGLGALPLHAQSLVGADRVSPSIHKLRQHRPLGRTQSAPLPQNAQALQHLVIQQQHQQFLEKHKQQFQQQQLHLNKILPKPSEPARQPESHPEETEEELREHQALLEEPFLDRLSGQKEVHALGVQVKQEPVESDDEEAEPPREAEPGQRQPREQELLFRQVLAYLILRTEETVFDQMPGPLGDKKAPVTAPQMRKSSMPRSPGSASALLEQQRIHQLRNYQASMEAAGVPVSFGGHRPLSRAQSSPASATFPMSVQEPPAKPRFTTGLVYDTLMLKHQCACGNTNSHPEHAGRIQSIWSRLQETGLRGKCECIRGRKATLEELQTVHSEAHALLYGTNPLNRQKLDSKKLLGSLTSVFVRLPCGGVGVDSDTIWSEVHSSGAARLAVGCVVELAFKVATGELKNGFAVVRPPGHHAEESTPMGFCYFNSVAIAAKLLQQRLHVSKTLVVDWDVHHGNGTQQAFYSDPSVLYVSLHRYDDGNFFPGSGAPDEVGTGPGVGFNVNMAFTGGLDPPMGDAEYLAAFRTVVMPIANEFAPDVVLVSSGFDAVEGHPTPLGGYNLSAKCFGYLTKQLMGLAGGRVVLALEGGHDLTAICDASEACVSALLGNELDPLPEKVLQQRPYDNSVRSMEKVIDIHSQYWRSLQRLASTVGYSLVEAQKCENEEAETVTAMASLSVGVKAPEKRPEEEPMEEEPPL

SEQ ID NO: 13 - protein, 5 aa, Artificial Sequence
  Description of Artificial Sequence Synthetic peptide
PXDLR

(19) United States
(12) Patent Application Publication
(10) Pub. No.: US 2016/0235826 A1
(43) Pub. Date: Aug. 18, 2016

(54) HISTONE DEACETYLASE COMPOSITIONS AND USES THEREOF

(71) Applicant: PRESIDENT AND FELLOWS OF HARVARD COLLEGE, Cambridge, MA (US)

(72) Inventors: Constance L. Cepko, Newton, MA (US); Bo Chen, Cambridge, MA (US); Wenjun Xiong, Cambridge, MA (US); Vedangi Sample, Boston, MA (US)

(21) Appl. No.: 15/022,301

(22) PCT Filed: Sep. 18, 2014
(86) PCT No.: PCT/US2014/056344

§ 371 Date: Mar. 16, 2016

Related U.S. Application Data
(60) Provisional application No. 61/879,292, filed on Sep. 18, 2013.

(51) Int. Cl. A61K 38/50; A61K 48/00; C12N 9/80; A61K 9/00

(52) U.S. Cl. CPC A61K 38/50; A61K 9/0048; A61K 48/0058; C12N 9/80; C12Y 305/01098

(57) ABSTRACT

The present invention is based, at least in part, on the discovery that fragments of the histone deacetylase 4 (HDAC4) gene lacking the enzymatic domain promoted rod survival when electroporated into the retinas of a relevant mouse model of retinitis pigmentosa. Specifically, it has been discovered that only a small portion of the N-terminus of HDAC4 promotes survival of rod cells in rd1 mice. Accordingly, the present invention provides histone deacetylase 4 compositions and methods of use thereof for inhibiting neuronal cell death, e.g., retinal cell death.

### RELATED APPLICATIONS

This application claims the benefit of priority to U.S. Provisional Patent Application No. 61/879,292, filed Sep. 18, 2013, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Sep. 17, 2014, is named 117823-04820_SL.txt and is 105,010 bytes in size.

### BACKGROUND OF THE INVENTION

Vision loss and blindness due to retinal cell death is a severe problem. In a 2004 publication, the World Health Organization (WHO) estimated that the global population of blind and visually impaired persons was over 300 million, with almost 10 million in the United States (WHO Bulletin 82 pp. 844-851—Global Data on Visual Impairment November, 2004).

Blindness is most often caused by loss of function and subsequent death of photoreceptor cells, the cells that initiate vision by capturing and transducing signals from light. Genetic lesions that directly affect photoreceptors are often responsible and there are now more than 200 disease genes in humans that lead to blindness (sph.uth.tmc.edu/Retnet). In addition, due to non-genetic as well as genetic causes, there are a large number of individuals over 75 years of age that lose their high acuity vision (Friedman, D. S., et al. (2004) Arch Ophthalmol 122:564-572). Retinal degenerative diseases, such as age-related macular degeneration (AMD) and retinitis pigmentosa (RP), are the most common cause of blindness (Friedman, D. S., et al. (2004) Arch Ophthalmol 122:564-572; Hartong, D. T., et al. (2006) Lancet 368:1795-1809).

As photoreceptor loss in mammals is irreversible, approaches to prevent cell death and/or replace or regenerate photoreceptors are the keys to treatments for these retinal degenerative diseases. However, most types of blindness have no effective treatment.

Thus, there is a need in the art for therapies to prevent, treat, diagnose and prognose vision loss that results from decreased retinal cell function.

### SUMMARY OF THE INVENTION

The present invention is based, at least in part, on the discovery that fragments of the histone deacetylase 4 (HDAC4) gene lacking the enzymatic domain promoted rod survival when electroporated into the retinas of a relevant mouse model of retinitis pigmentosa. Specifically, it has been discovered that only a small portion of the N-terminus of HDAC4 promotes survival of rod cells in rd1 mice. Accordingly, the present invention provides histone deacetylase 4 compositions and methods of use thereof for inhibiting neuronal cell death, e.g., retinal cell death.

In one aspect, the present invention provides pharmaceutical compositions suitable for intraocular administration which comprise, consist essentially of, or consist of, an isolated HDAC4 nucleic acid molecule comprising nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1. In one embodiment, a therapeutically or prophylactically effective amount of the nucleic acid molecule is contained in the compositions suitable for intraocular administration.

In another aspect, the present invention provides pharmaceutical compositions suitable for intraocular administration which comprise, consist essentially of, or consist of, an isolated HDAC4 nucleic acid molecule encoding a peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2. In one embodiment, a therapeutically or prophylactically effective amount of the nucleic acid molecule is contained in the compositions suitable for intraocular administration.

In yet another aspect, the present invention provides pharmaceutical compositions suitable for intraocular administration which comprise, consist essentially of, or consist of, an isolated HDAC4 peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2, suitable for intraocular administration. In one embodiment, a therapeutically or prophylactically effective amount of the peptide is contained in the compositions suitable for intraocular administration.

In another aspect, the present invention provides isolated HDAC4 nucleic acid molecules comprising, consisting essentially of, or consisting of, nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1.

In another aspect, the present invention provides isolated HDAC4 nucleic acid molecules encoding a peptide comprising, consisting essentially of, or consisting of, amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2.

In one embodiment, the peptide is localized to the cytoplasm of a neuronal cell. In one embodiment, the peptide inhibits neuronal cell death. The cell may be a retinal cell, such as a bipolar cell, a rod photoreceptor cell and/or a cone photoreceptor cell.

In one aspect, the present invention provides vectors, e.g., expression vectors, comprising the isolated nucleic acid molecules of the invention.

In one embodiment, the vector is a viral vector, e.g., selected from the group consisting of a retroviral vector, an adenoviral vector, an adenovirallretroviral chimera vector, an adeno-associated viruus (AAV) vector, a herpes simplex viral I or II vector, a parvovirus vector, a reticuloendotheliosis virus vector, a poliovirus vector, a papillomavirus vector, a vaccinia virus vector, and a lentivirus vector. In one embodiment, the vector is an AAV vector, e.g., an AAV 2/5 or an AAV 2/8 vector.

In one aspect, the present invention provides viral vectors comprising a retinal cell-type specific promoter operably linked to an HDAC4 nucleic acid molecule comprising, consisting essentially of, or consisting of, nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1.

In another aspect, the present invention provides viral vectors comprising a retinal cell-type specific promoter operably linked to a nucleic acid molecule encoding an HDAC4 peptide comprising, consisting essentially of, or consisting of, amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2.

The retinal cell-type specific promoter may be a rod-specific promoter, a cone-specific promoter, and/or a rod- and cone-specific promoter.

In one embodiment, the composition is suitable for intraocular administration, e.g., sub-retinal or intravitreal administration.

In one aspect, the present invention provides methods of inhibiting neuronal cell, e.g., retinal cell, death. The methods include contacting the cell with an isolated HDAC4 nucleic acid molecule comprising, consisting essentially of, or consisting of, nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1, thereby inhibiting death of the neuronal cell.

In another aspect, the present invention provides methods for treating or preventing a neurodegenerative disorder, e.g., retinitis pigmentosa, in a subject. The methods include administering to the subject an isolated HDAC4 nucleic acid molecule comprising, consisting essentially of, or consisting of, nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1, thereby treating or preventing the neurodegenerative disorder in the subject.

In one aspect, the present invention provides methods of inhibiting neuronal cell, e.g., retinal cell, death. The methods include contacting the cell with an isolated nucleic acid molecule encoding an HDAC4 peptide comprising, consisting essentially of, or consisting of, amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2, thereby inhibiting death of the neuronal cell.

In another aspect, the present invention provides methods for treating or preventing a neurodegenerative disorder, e.g., retinitis pigmentosa, in a subject. The methods include administering to the subject an isolated nucleic acid molecule encoding an HDAC4 peptide comprising, consisting essentially of, or consisting of, amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2, thereby treating or preventing the neurodegenerative disorder in the subject.

In one aspect, the present invention provides methods of inhibiting neuronal cell, e.g., retinal cell death. The methods include contacting the cell with an isolated HDAC4 peptide comprising, consisting essentially of, or consisting of, amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2, thereby inhibiting death of the neuronal cell.

In another aspect, the present invention provides methods for treating or preventing a neurodegenerative disorder, e.g., retinitis pigmentosa, in a subject. The methods include administering to the subject an isolated HDAC4 peptide comprising, consisting essentially of, or consisting of, amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2, thereby treating or preventing said neurodegenerative disorder in the subject.

In one embodiment, the nucleic acid molecule encodes a peptide localized to the cytoplasm of the neuronal cell. In one embodiment, the peptide is localized to the cytoplasm of the neuronal cell. In one embodiment, the peptide inhibits neuronal cell death.

The cell may be a retinal cell, such as a bipolar cell, a rod photoreceptor cell and/or a cone photoreceptor cell.

In one embodiment, the neuronal cell death is naturally occurring. In one embodiment, the neuronal cell death is caused by a neurodegenerative disorder, e.g., age-related macular degeneration, or retinitis pigmentosa.

In one embodiment, the nucleic acid molecule is contained within a vector, e.g., an expression vector, comprising the isolated nucleic acid molecules of the invention

In one embodiment, the vector is a viral vector, e.g., selected from the group consisting of a retroviral vector, an adenoviral vector, an adenovirallretroviral chimera vector, an adeno-associated viruus (AAV) vector, a herpes simplex viral I or II vector, a parvovirus vector, a reticuloendotheliosis virus vector, a poliovirus vector, a papillomavirus vector, a vaccinia virus vector, and a lentivirus vector. In one embodiment, the vector is an AAV vector, e.g., an AAV 2/5 or an AAV 2/8 vector.

In one embodiment, the vector comprises a retinal cell-type specific promoter, e.g., a rod-specific promoter, a cone-specific promoter, and/or a rod- and cone-specific promoter, operably linked to the nucleic acid molecule.

In one embodiment, the contacting is within the eye of a subject.

In one embodiment, the administration is intraocular administration, e.g., sub-retinal or intravitreal administration.

### BRIEF DESCRIPTION OF THE DRAWINGS

FIGS. 1A-1F depict AAV infection and expression of AAV-CMV-GFP in rods and cones of wild type (WT) and retinal degeneration 1 (rd1) mice retinas. (A). A WT retina was infected at P0 with AAV-CMVGFP. A low magnification image of a cryosection shows extensive spread of the infection throughout the retina. (B). A higher magnification view of the outer nuclear layer in A, showing that cones are well infected and express GFP at a high level (anti-GFP in light gray, PNA (a cone marker) in medium gray). Bracket indicates entirety of cones, with top arrow pointing to cone outer segment (medium gray PNA stain), middle arrow to cone inner segment and bottom arrow to cone cell body (C). Same as A but an rd1 retina was infected. (D). Higher magnification view of C, with remaining cones at P30 well stained with anti-GFP (light gray) and PNA (medium gray). Arrow points to a single cone, and bracket indicates layer of cone cell bodies. (E). Rods from the retina shown in A, visualized by a longer exposure to show that they are infected but express at a lower level (compare to (B), with arrow pointing to a cone. (F). Flat mount image of an rd1 retina infected with AAV-CMV-GFP at P0. A 60× (250 micron square area) image taken 1 mm from the optic nerve head, with the focal plane in the remaining outer nuclear layer is shown.

FIG. 2 is a graph depicting the rescue of rod photoreceptors in the rd1 RP mouse model. Constructs with full length HDAC4 comprising the enzymatic deacetylase domain (fl) (e.g., nucleotides 793-8459 of SEQ ID NO:1 or a nucleotide sequence encoding amino acid residues 1-1,084 of SEQ ID NO:2); a fragment of HDAC4 comprising nucleotides 978-1257 of SEQ ID NO:1 (or, a nucleotide sequence encoding amino acid residues 62-155 of SEQ ID NO:2) (poly Q); a fragment of HDAC4 comprising nucleotides 793-1170 of SEQ ID NO:1 (or, a nucleotide sequence encoding amino acid residues 1-126 of SEQ ID NO:2) (stop); no HDAC 4 (control); and HIFlalpha DN which is a dominant negative allele that blocks transcription of HIFlalpha targets were electroporated into the mouse retina at P0. At P60, the number of rods was quantified per a defined area of the retina.

FIG. 3 is a schematic of an AAV vector used to deliver HDCA4 nucleic acid molecules and peptides. The vector may comprise the following elements; an inverted terminal repeat (ITR) from, e.g., an AAV2; a promoter. e.g., a promoter from a photoreceptor-specific gene; a β-globin intron to, e.g., provide for splicing of the transcript; an HDAC4 coding sequence; a woodchuck post-transciptional response element (WPRE) to, e.g., provide for higher level gene expression; and an SV40 polyA element for, e.g., polyadenylation of an mRNA.

FIG. 4 is a schematic of a full-length HDAC4 protein showing its multiple binding domains. FIG. 4 also provides a schematic of two deletion alleles of HDAC4 that include only amino acid residues 1-208 or amino acid residues 1-118 used to prepare constructs that were electroporated into the retinas of a mouse model of RP, and were shown to be effective for rod rescue.

FIG. 5 is a graph depicting the rescue of rod photoreceptors in the rd1 RP mouse model. Constructs of full length HDAC4 (FL-HDAC4); a peptide fragment of HDAC4 comprising amino acids 1-118 (118aaAU1); a peptide fragment of HDAC4 comprising amino acids 1-208 (208aaAU1); HDAC6 (control); and GFP (control) were electroporated into the mouse retina at postnatal day 0. Astericks indicate statistical significance, with p-values shown for the indicated comparisons. The p-values are as indicated.

FIGS. 6A-D depict expression of full-length HDAC4 and HDAC4 in rd mouse retinas. Constructs comprising GFP alone (control) (A), HDAC5 control (B) and a fragment of HDAC4 comprising amino acids 1-118 (118aaAU1) (C) were electroporated into the mouse retina at postnatal day 0. Retina from infected mice were removed and prepared as flat mounts at postnatal day 50. Light gray indicates nuclei (DAPI stain), dark gray indicates expression of CAG-GFP (or electroporated area) and medium gray indicates surviving rods (expression of Rho-DsRed). The presence of surviving was assayed by immunohistochemistry for HDCA4. (D) A higher magnification view of retinas electroporated with the indicated constructs.

### DETAILED DESCRIPTION OF THE INVENTION

Histone deacetylases (HDAC) are involved in the control of histone acetylation status and form a group of 18 proteins divided into two major families: the zinc-dependent hydrolases, arranged into class I, II, and IV, and the evolutionarily distinct, NAD-dependent, sirtuin-like class III proteins (Roth S Y, et al. (2001) Annu Rev Biochem 70: 81-120; Marks P A, et al. (2003) Curr Opin Pharmacol 3: 344-51; and Gallinari P, et al. (2007) Cell Res 17: 195-211). HDACs deacetylate histone proteins leading to a repression of transcription. One of the Class I HDACs, HDAC4 is involved in the control of gene expression through its recruitment by transcription factors, notably of the Mef (e.g., Mef2) and Runx families, or by transcriptional co-repressor complexes such as CtBP (Verdin E, et al. (2003) Trends Genet 19: 286-93; Zhang C L et al. (2001) J Biol Chem276: 35-9; Vega R B, et al. (2004) Cell 119: 555-66). HDAC4 also associates with heterochromatin via direct binding to HP1 and thus participates in higher-order structures, possibly contributing to the propagation of repressive chromate (Verdin E, et al. (2003) Trends Genet 19: 286-93). In addition, the deacetylase domain of HDAC4 interacts with, e.g., HDAC3 and HDAC1, via the transcriptional corepressor NCOR2 to repress transcription.

The present invention is based, at least in part, on the surprising and unexpected discovery that fragments of the histone deacetylase 4 (HDAC4) gene lacking the enzymatic domains responsible for its function as a transcriptional repressor promoted rod survival when electroporated into the retinas of a relevant mouse model of retinitis pigmentosa. Specifically, it has been discovered that only a small portion of the N-terminus of HDAC4 promotes survival of rod cells in rd1 mice. Accordingly, the present invention provides histone deacetylase 4 compositions and methods of use thereof for inhibiting photoreceptor death.

### I. DEFINITIONS

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the term “neurodegenerative disorder” includes, but is not limited to, neurodegenerative disorders of the eye such as ocular disorders. In one embodiment, a neurodegenerative disorder is an ocular disorder that is the result of loss of function and subsequent death of photoreceptor cells. Non-limiting examples of ocular disorders include macular degeneration (e.g., atrophic and age-related), retinitis pigmentosa, iatrogenic retinopathy, retinal tears and holes, diabetic retinopathy, sickle cell retinopathy, retinal vein and artery occlusion and the like. In various embodiments, the ocular disorder is associated with decreased viability of cone and/or rod cells. In another embodiment, the ocular disorder is a genetic disorder. In yet another embodiment, the ocular disorder is not diabetic retinopathy. Alternatively or in addition, the ocular disorder is not associated with blood vessel leakage and/or growth.

The term “neurodegenerative disorder” also includes, but is not limited to, certain ophthalmic disorders, such as sickle cell retinopathy and retinal vein or artery occlusion, that can be characterized by both angiogenesis and neurodegenerative components.

The term “neurodegenerative disorder” further includes disorders such as optic neuropathy, Alzheimer's disease, Parkinson's disease, Huntington's disease, Pick's disease, Pelizaeus-Merzbacher disease, Resfum's disease, Sandhoff disease, Schilder's disease, Steele-Richardson-Olszewski disease, amyotrophic lateral sclerosis, primary lateral sclerosis, multiple sclerosis, multiple system atrophy, narcolepsy, neuroborreliosis, spinocerebellar ataxia, spinal muscular atrophy, tabes dorsalis, prion diseases (e.g., scrapie, Creutzfeldt-Jakob disease, Gerstmann-Strassler Scheinker disease, bovine spongiform encephalopathy and the like), Alexander disease, Alper's disease, ataxia telangiectasia, Batten disease, Canavan disease, Cockayne syndrome, corticobasal degeneration, HIV-associated dementia, Kennedy's disease, Krabbe disease, Lewy body dementia, spinocerebellar ataxia type 3, subacute combined degeneration of spinal cord secondary to pernicious anemia, schizophrenia, Batten disease and the like.

A “patient” or “subject,” as used herein, is intended to include either a human or non-human animal, preferably a mammal, e.g., human or a monkey. Most preferably, the subject or patient is a human.

In one embodiment of the invention, cells suitable for use in the instant methods are neuronal cells. As used herein, the terms “neuron” or “neuronal cell” refer to a nerve cell capable of receiving and conducting electrical impulses from the nervous system. A nerve cell or “neuron” typically comprises a cell body, an axon, axon terminals, and dendrites and is readily identifiable by one of ordinary skill in the art.

In one embodiment, a neuron is a “photoreceptor cell”, i.e., a specialized neuron found in the retina. The retina is a thin, transparent tissue containing about 120 million separate rod cells (night vision) and 7 million cone cells (day and color vision) as well as millions of other structural supporting and interconnecting cells. Photoreceptor cells consist of “rods” and “cones”, which are the photosensitive cells of the retina. The rods contain rhodopsin, the rod photopigment, and the cones contain other distinct photopigments, which respond to light and trigger a neural discharge in the output cells of the retina, the ganglion cells. Ultimately, this signal is registered as a visual stimulus in the visual cortex and other target locations in the brain. The retinal pigment epithelial (RPE) cells produce, store and transport a variety of factors that are responsible for the normal function and survival of photoreceptors. Retinal neurons that can also sense light consist of photosensitive ganglion cells. These cells, known as the melanopsin ganglion cells are found in the inner retina, have dendrites and long axons projecting to the protectum (midbrain), the suprachiasmatic nucleas in the hypothalamus, and the lateral geniculate (thalamus). The retina also consists of bipolar cells which exist between photoreceptors (rod cells and cone cells) and ganglion cells. These cells transmit signals from the photoreceptors to the ganglion cells. Bipolar cells receive synaptic input from either rods or cones, but not both, and they are designated rod bipolar or cone bipolar cells respectively. In one embodiment, a photoreceptor cell is a rod. In one embodiment, a photoreceptor cell is a cone. In one embodiment, a photoreceptor cell is a cell is a bipolar cell.

As used herein, the term “contacting” (i.e., contacting a cell with an agent) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

As used herein, “histone deacetylase 4” or “HDAC4” (also known as KIAA02881, HD4, EC 3.5.1.98, HDACA, AHO3, HA6116, BDMR, HDAC-4, Histone Deacetylase A, and HDAC-A) refers to the Class II histone deacetylase which catalyzes the removal of acetyl groups from lysine residues in histones and non-histone proteins, resulting in, for example, transcriptional repression. The term includes human HDAC4, the nucleotide and amino acid sequence of which may be found in, for example, GenBank Accession No. GI:153085394 (SEQ ID NOs:1 and 2, respectively); mouse HDAC4, the nucleotide and amino acid sequence of which may be found in, for example, GenBank Accession No. GI: 46402200 (SEQ ID NOs:3 and 4, respectively); dog HDAC4, the nucleotide and amino acid sequence of which may be found in, for example, GenBank Accession No. GI:345790779 (SEQ ID NOs:5 and 6, respectively); rat HDAC4, the nucleotide and amino acid sequence of which may be found in, for example, GenBank Accession No. GI:402744247 (SEQ ID NOs:7 and 8, respectively); chimpanzee HDAC4, the nucleotide and amino acid sequence of which may be found in, for example, GenBank Accession No. GI:332815844 (SEQ ID NOs:9 and 10, respectively); and pig HDAC4, the nucleotide sequence and amino acid of which may be found in, for example, GenBank Accession No. GI:350594075 (SEQ ID NOs:11 and 12). Additional examples of HDAC4 sequences are readily available using, e.g., GenBank.

### II. HISTONE DEACETYLASE COMPOSITIONS

In one aspect, the present invention provides isolated HDCA4 nucleic acid molecules, e.g., nucleic acid molecules comprising fragments of an HDAC4 gene. Such nucleic acid molecules encode N-terminal HDAC4 peptides that lack the HDAC4 enzymatic domain (e.g., deacetylase domain). The isolated nucleic acid molecules of the invention encode peptides that are localized to the cytoplasm of a neuronal cell, e.g., a retinal cell, inhibit neuronal, e.g., retinal, cell death, and/or are useful for, e.g., treating neurodegenerative disorders (described below).

Accordingly, in one aspect, the present invention provides isolated nucleic acid molecules comprising nucleotides 793-1416 of SEQ ID NO:1. In one embodiment, the isolated nucleic acid molecules comprise nucleotides 793-1170 of SEQ ID NO:1. In another embodiment, the isolated nucleic acid molecules comprise nucleotides 793-1146 of SEQ ID NO:1. In yet another embodiment, the isolated nucleic acid molecules comprise nucleotides 978-1257 of SEQ ID NO:1.

In another aspect, the present invention provides isolated nucleic acid molecules comprising nucleotides 117-737 of SEQ ID NO:3. In one embodiment, the isolated nucleic acid molecules comprise nucleotides 117-491 of SEQ ID NO:3. In another embodiment, the isolated nucleic acid molecules comprise nucleotides 117-467 of SEQ ID NO:3. In yet another embodiment, the isolated nucleic acid molecules comprise nucleotides 302-578 of SEQ ID NO:3.

In another aspect, the present invention provides isolated nucleic acid molecules comprising nucleotides 1-690 of SEQ ID NO:5. In one embodiment, the isolated nucleic acid molecules comprise nucleotides 1-444 of SEQ ID NO:5. In another embodiment, the isolated nucleic acid molecules comprise nucleotides 1-420 of SEQ ID NO:5. In yet another embodiment, the isolated nucleic acid molecules comprise nucleotides 252-531 of SEQ ID NO:5.

In another aspect, the present invention provides isolated nucleic acid molecules comprising nucleotides 296-916 of SEQ ID NO:7. In one embodiment, the isolated nucleic acid molecules comprise nucleotides 296-670 of SEQ ID NO:7. In another embodiment, the isolated nucleic acid molecules comprise nucleotides 296-646 of SEQ ID NO:7. In yet another embodiment, the isolated nucleic acid molecules comprise nucleotides 481-757 of SEQ ID NO:7.

In another aspect, the present invention provides isolated nucleic acid molecules comprising nucleotides 837-1460 of SEQ ID NO:9. In one embodiment, the isolated nucleic acid molecules comprise nucleotides 837-1214 of SEQ ID NO:9. In another embodiment, the isolated nucleic acid molecules comprise nucleotides 837-1190 of SEQ ID NO:9. In ye another embodiment, the isolated nucleic acid molecules comprise nucleotides 1022-1301 of SEQ ID NO:9.

In another aspect, the present invention provides isolated nucleic acid molecules comprising nucleotides 1-1797 of SEQ ID NO:11. In one embodiment, the isolated nucleic acid molecules comprise nucleotides 1-1551 of SEQ ID NO:11. In another embodiment, the isolated nucleic acid molecules comprise nucleotides 1-1527 of SEQ ID NO:11. In yet another embodiment, the isolated nucleic acid molecules comprise nucleotides 1359-1638 of SEQ ID NO:11.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

In one aspect of the invention, an isolated nucleic acid molecule of the invention is a nucleic acid molecule encoding HDAC4 peptides, e.g., an N-terminal HDAC4 peptide that lacks the HDAC4 enzymatic domain (e.g., deacetylase domain).

For example, nucleic acid molecules encoding an HDAC4 peptide of interest in the form suitable for expression of the protein in a host cell can be prepared using nucleotide sequences based on the nucleotide sequence encoding an HDAC4 peptide of interest. For example, a cDNA (full length or partial cDNA sequence) may be cloned into a vector, such as a recombinant expression vector, and the vector may be transfected into cells using standard molecular biology techniques. The cDNA can be obtained, for example, by amplification using the polymerase chain reaction (PCR) or by screening an appropriate cDNA library.

In one embodiment, the isolated peptides and/or isolated nucleic acid molecules encode peptides that comprise the binding motif, P-X-D-L-R (SEQ ID NO:13) (e.g., amino acid residues 43-57 of SEQ ID NO:2) and bind the transcriptional co-repressor, COOH-terminal-binding protein (CtBP) (e.g., through amino acid residues 43-57 of SEQ ID NO:2) (see, e.g., Zhang, C. L., et al. (2001) JBC 276:35-39). In another embodiment, the isolated nucleic acid molecules encode peptides that do not bind CtBP through a CtBP-binding motif (P-X-D-L-R) (SEQ ID NO:13).

In one embodiment, the isolated peptides and/or isolated nucleic acid molecules encode peptides that bind Runt-related transcription factor 2 (RUNX2) also known as core-binding factor subunit alpha-1 (CBF-alpha-1) and/or histone deacetylace 1 (HDAC1) (e.g., through amino acid residues 118-208 of SEQ ID NO:2) (see, e.g., Chan, J. K., et al. (2003) JBC 278:23515-23521). In another embodiment, the isolated nucleic acid molecules encode peptides that do not bind RUNX2 and/or HDAC1.

In one embodiment, isolated peptides and/or the isolated nucleic acid molecules encode peptides that bind myocyte enhancer factor 2C (MEF2C), also known as MADS box transcription enhancer factor 2, polypeptide C (e.g., through amino acid residues 163-184 of SEQ ID NO:2) (see, e.g., Chan, J. K., et al. (2003) JBC 278:23515-23521). In another embodiment, the isolated nucleic acid molecules encode peptides that do not bind MEF2C.

Accordingly, in one aspect the present invention provides isolated nucleic acid molecules encoding a peptide comprising amino acid residues 1-208 of SEQ ID NO:2. In one embodiment, the isolated nucleic acid molecules encode a peptide comprising amino acid residues 1-126 of SEQ ID NO:2. In another embodiment, the isolated nucleic acid molecules encode a peptide comprising amino acid residues 1-118 of SEQ ID NO:2. In yet another embodiment, the isolated nucleic acid molecules encode a peptide comprising amino acid residues 62-155 of SEQ ID NO:2.

In another aspect, the present invention provides isolated nucleic acid molecules encoding a peptide comprising amino acid residues 1-207 of SEQ ID NO:4. In one embodiment, the isolated nucleic acid molecules encode a peptide comprising amino acid residues 1-125 of SEQ ID NO:4. In another embodiment, the isolated nucleic acid molecules encode a peptide comprising amino acid residues 1-117 of SEQ ID NO:4. In yet another embodiment, the isolated nucleic acid molecules encode a peptide comprising amino acid residues 62-154 of SEQ ID NO:4.

In another aspect, the present invention provides isolated nucleic acid molecules encoding a peptide comprising amino acid residues 1-230 of SEQ ID NO:6. In one embodiment, the isolated nucleic acid molecules encode a peptide comprising amino acid residues 1-148 of SEQ ID NO:6. In another embodiment, the isolated nucleic acid molecules encode a peptide comprising amino acid residues 1-140 of SEQ ID NO:6. In yet another embodiment, the isolated nucleic acid molecules encode a peptide comprising amino acid residues 84-177 of SEQ ID NO:6.

In another aspect, the present invention provides isolated nucleic acid molecules encoding a peptide comprising amino acid residues 1-207 of SEQ ID NO:8. In one embodiment, the isolated nucleic acid molecules encode a peptide comprising amino acid residues 1-125 of SEQ ID NO:8. In another embodiment, the isolated nucleic acid molecules encode a peptide comprising amino acid residues 1-117 of SEQ ID NO:8. In yet another embodiment, the isolated nucleic acid molecules encode a peptide comprising amino acid residues 62-154 of SEQ ID NO:8.

In another aspect, the present invention provides isolated nucleic acid molecules encoding a peptide comprising amino acid residues 1-208 of SEQ ID NO:10. In one embodiment, the isolated nucleic acid molecules encode a peptide comprising amino acid residues 1-126 of SEQ ID NO:10. In another embodiment, the isolated nucleic acid molecules encode a peptide comprising amino acid residues 1-118 of SEQ ID NO:10. In yet another embodiment, the isolated nucleic acid molecules encode a peptide comprising amino acid residues 62-155 of SEQ ID NO:10. In another aspect, the present invention provides isolated nucleic acid molecules encoding a peptide comprising amino acid residues 1-599 of SEQ ID NO:12. In one embodiment, the isolated nucleic acid molecules encode a peptide comprising amino acid residues 1-517 of SEQ ID NO:12. In another embodiment, the isolated nucleic acid molecules encode a peptide comprising amino acid residues 1-509 of SEQ ID NO:12. In yet another embodiment, the isolated nucleic acid molecules encode a peptide comprising amino acid residues 453-546 of SEQ ID NO:12.

By virtue of the nature of the nucleotide and amino acid sequences provided herein, it is contemplated that for any sequence identified herein, e.g., nucleotides 793-1416 of SEQ ID NO:1, nucleotides 793-1170 of SEQ ID NO:1, nucleotides 793-1146 of SEQ ID NO:1, nucleotides 978-1257 of SEQ ID NO:1, amino acid residues 1-208 of SEQ ID NO:2, amino acid residues 1-126 of SEQ ID NO:2, amino acid residues 1-118 of SEQ ID NO:2, and amino acid residues 62-155 of SEQ ID NO:2, optimization of any of these sequence could be achieved by systematically either adding or removing nucleotides or amino acid residues to generate longer or shorter sequences and testing those sequences generated by walking a window of the longer or shorter size up or down the identified region from that point. Coupling this approach to generating new candidate nucleic acid molecules and/or peptides with testing for effectiveness for rod rescue as known in the art and/or as described herein can lead to further improvements in the efficiency of photoreceptor survival.

Thus, for example, the present invention includes isolated nucleic acid molecules and peptides comprising, one, two, three, or four nucleotides at the 5′-end and/or the 3′-end of the nucleotide sequences identified herein, or one, two, three, or four amino acid residues at the N-terminus or C-terminus of the peptide sequences identified herein (e.g., amino acid residues 1-118 of SEQ ID NO:2). The additional nucleotides or amino acid residues may be additional HDAC4 nucleotides or amino acid residues (e.g., amino acid residues 119, 120, 121, or 121 of SEQ ID NO:2 may be added to the C-terminus of a peptide comprising amino acid residues 1-118 of SEQ ID NO:2) or the additional nucleotides or amino acid residues may be non-HDAC4 nucleotides or amino acid residues (heterologous nucleotides or amino acid residues) operably linked or fused to the sequences disclosed herein. Such additional nucleotides or amino acid residues may serve to, e.g., improve stability, effectiveness, or potency of the nucleic acid molecule or peptide to which they are attached. The term “operably linked” is intended to indicate that the sequences disclosed herein and the heterologous nucleotides or amino acid residues are fused in-frame to each other.

In one embodiment, the nucleic acid molecules of the invention may be delivered to cells, e.g., neuronal cells, or to subjects, in a vector, e.g., a recombinant expression vector. In another embodiment, the nucleic acid molecules of the invention may be delivered to cells, e.g., neuronal cells, or to subjects, in the absence of a vector.

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention (e.g., a nucleic acid sequence encoding an HDAC4 petide) in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells, those which are constitutively active, those which are inducible, and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or portions thereof, including fusion proteins or portions thereof, encoded by nucleic acids as described herein.

Recombinant expression vectors of the invention can be designed for expression of the nucleic acid molecules of the invention in prokaryotic or eukaryotic cells. For example, an HDAC4 peptide can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40); pMAL (New England Biolabs, Beverly, Mass.); and pRITS (Pharmacia, Piscataway, N.J.) which fuse glutathione S-transferase (GST), maltose E binding protein, or protein A, respectively, to the target recombinant protein.

In another embodiment, the expression vector is a yeast expression vector. Examples of vectors for expression in yeast S. cerevisiae include pYepSec1 (Baldari, et. al., (1987) EMBO J. 6:229-234); pMFa (Kurjan and Herskowitz, (1982) Cell 30:933-943); pJRY88 (Schultz et al., (1987) Gene 54:113-123); pYES2 (Invitrogen Corporation, San Diego, Calif.); and picZ (Invitrogen Corporation).

Alternatively, HDAC4 peptides can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements.

In certain embodiments, the nucleic acid molecules of the invention are contained within a viral vector and may be delivered to cells, e.g., neuronal cells, or to subjects. Preferably a viral vector is one whose use for gene therapy is well known in the art. Techniques for the formation of vectors or virions are generally described in “Working Toward Human Gene Therapy,” Chapter 28 in Recombinant DNA, 2nd Ed., Watson, J. D. et al., eds., New York: Scientific American Books, pp. 567-581 (1992). An overview of suitable viral vectors or virions is provided in Wilson, J. M., Clin. Exp. Immunol. 107(Suppl. 1):31-32 (1997), as well as Nakanishi, M., Crit. Rev. Therapeu. Drug Carrier Systems 12:263-310 (1995); Robbins, P. D., et al., Trends Biotechnol. 16:35-40 (1998); Zhang, J., et al., Cancer Metastasis Rev. 15:385-401(1996); and Kramm, C. M., et al., Brain Pathology 5:345-381 (1995). Such vectors may be derived from viruses that contain RNA (Vile, R. G., et al., Br. Med Bull. 51:12-30 (1995)) or DNA (Ali M., et al., Gene Ther. 1:367-384 (1994)).

Examples of viral vector systems utilized in the gene therapy art and, thus, suitable for use in the present invention, include the following: retroviruses (Vile, R. G., supra; U.S. Pat. Nos. 5,741,486 and 5,763,242); adenoviruses (Brody, S. L., et al., Ann. N.Y. Acad. Sci. 716: 90-101 (1994); Heise, C. et al., Nat. Med. 3:639-645 (1997)); adenoviral/retroviral chimeras (Bilbao, G., et al., FASEB J. 11:624-634 (1997); Feng, M., et al., Nat. Biotechnol. 15:866-870 (1997)); adeno-associated viruses (Flotte, T. R. and Carter, B. J., Gene Ther. 2:357-362 (1995); U.S. Pat. No. 5,756,283); herpes simplex virus I or II (Latchman, D. S., Mol. Biotechnol. 2:179-195 (1994); U.S. Pat. No. 5,763,217; Chase, M., et al., Nature Biotechnol. 16:444-448 (1998)); parvovirus (Shaughnessy, E., et al., Semin Oncol. 23:159-171 (1996)); reticuloendotheliosis virus (Donburg, R., Gene Therap. 2:301-310 (1995)). Extrachromosomal replicating vectors may also be used in the gene therapy methods of the present invention. Such vectors are described in, for example, Calos, M. P. (1996) Trends Genet. 12:463-466, the entire contents of which are incorporated herein by reference. Other viruses that can be used as vectors for gene delivery include poliovirus, papillomavirus, vaccinia virus, lentivirus, as well as hybrid or chimeric vectors incorporating favorable aspects of two or more viruses (Nakanishi, M. (1995) Crit. Rev. Therapeu. Drug Carrier Systems 12:263-310; Zhang, J., et al. (1996) Cancer Metastasis Rev. 15:385-401; Jacoby, D. R., et al. (1997) Gene Therapy 4:1281-1283).

As used herein, the term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules which encode the structural proteins and enzymes needed to produce new viral particles. At each end of the provirus are structures called “long terminal repeats” or “LTRs.” LTRs contain numerous regulatory signals, including transcriptional control elements, polyadenylation signals, and sequences needed for replication and integration of the viral genome. LTRs may be several hundred base pairs in length.

The term “AAV vector” refers to a vector derived from an adeno-associated virus serotype, including without limitation, AAV-1, AAV-2, AAV-3, AAV-4, AAV-5, or AAVX7. “rAAV vector” refers to a vector that includes AAV nucleotide sequences as well as heterologous nucleotide sequences. rAAV vectors require only the 145 base terminal repeats in cis to generate virus. All other viral sequences are dispensable and may be supplied in trans (Muzyczka (1992) Curr. Topics Microbiol. Immunol. 158:97). Typically, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging. In particular embodiments, the AAV vector is an AAV2/5 or AAV2/8 vector. Suitable AAV vectors are described in, for example, U.S. Pat. No. 7,056,502 and Yan et al. (2002) J. Virology 76(5):2043-2053, the entire contents of which are incorporated herein by reference.

As used herein, the term “lentivirus” refers to a group (or genus) of retroviruses that give rise to slowly developing disease. Viruses included within this group include HIV (human immunodeficiency virus; including but not limited to HIV type 1 and HIV type 2), the etiologic agent of the human acquired immunodeficiency syndrome (AIDS); visna-maedi, which causes encephalitis (visna) or pneumonia (maedi) in sheep; the caprine arthritis-encephalitis virus, which causes immune deficiency, arthritis, and encephalopathy in goats; equine infectious anemia virus (EIAV), which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SW), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells). In one embodiment of the invention, the lentivirus is not HIV.

As used herein, the term “adenovirus” (“Ad”) refers to a group of double-stranded DNA viruses with a linear genome of about 36 kb. See, e.g., Berkner et al., Curr. Top. Microbiol. Immunol., 158: 39-61 (1992). In some embodiments, the adenovirus-based vector is an Ad-2 or Ad-5 based vector. See, e.g., Muzyczka, Curr. Top. Microbiol. Immunol., 158: 97-123, 1992; Ali et al., 1994 Gene Therapy 1: 367-384; U.S. Pat. Nos. 4,797,368, and 5,399,346. Suitable adenovirus vectors derived from the adenovirus strain Ad type 5 d1324 or other strains of adenovirus (e.g., Ad2, Ad3, Ad7, etc.) are well known to those skilled in the art. Recombinant adenoviruses are advantageous in that they do not require dividing cells to be effective gene delivery vehicles and can be used to infect a wide variety of cell types. Additionally, introduced adenovirus DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenovirus genome for foreign DNA is large (up to 8 kilobases) relative to other gene delivery vectors (Haj-Ahmand et al. J. Virol. 57, 267-273 [1986]).

In one embodiment, an adenovirus is a replication defective adenovirus. Most replication-defective adenoviral vectors currently in use have all or parts of the viral E1 and E3 genes deleted but retain as much as 80% of the adenovirus genetic material. Adenovirus vectors deleted for all viral coding regions are also described by Kochanek et al. and Chamberlain et al. (U.S. Pat. No. 5,985,846 and U.S. Pat. No. 6,083,750). Such viruses are unable to replicate as viruses in the absence of viral products provided by a second virus, referred to as a “helper” virus.

In one embodiment, an adenoviral vector is a “gutless” vector. Such vectors contain a minimal amount of adenovirus DNA and are incapable of expressing any adenovirus antigens (hence the term “gutless”). The gutless replication defective Ad vectors provide the significant advantage of accommodating large inserts of foreign DNA while completely eliminating the problem of expressing adenoviral genes that result in an immunological response to viral proteins when a gutless replication defective Ad vector is used in gene therapy. Methods for producing gutless replication defective Ad vectors have been described, for example, in U.S. Pat. No. 5,981,225 to Kochanek et al., and U.S. Pat. Nos. 6,063,622 and 6,451,596 to Chamberlain et al; Parks et al., PNAS 93:13565 (1996) and Lieber et al., J. Virol. 70:8944-8960 (1996).

In another embodiment, an adenoviral vector is a “conditionally replicative adenovirus” (“CRAds”). CRAds are genetically modified to preferentially replicate in specific cells by either (i) replacing viral promoters with tissue specific promoters or (ii) deletion of viral genes important for replication that are compensated for by the target cells only. The skilled artisan would be able to identify epithelial cell specific promoters.

Other art known adenoviral vectors may be used in the methods of the invention. Examples include Ad vectors with recombinant fiber proteins for modified tropism (as described in, e.g., van Beusechem et al., 2000 Gene Ther. 7: 1940-1946), protease pre-treated viral vectors (as described in, e.g., Kuriyama et al., 2000 Hum. Gene Ther. 11: 2219-2230), E2a temperature sensitive mutant Ad vectors (as described in, e.g., Engelhardt et al., 1994 Hum. Gene Ther. 5: 1217-1229), and “gutless” Ad vectors (as described in, e.g., Armentano et al., 1997 J. Virol. 71: 2408-2416; Chen et al., 1997 Proc. Nat. Acad. Sci. USA 94: 1645-1650; Schieder et al., 1998 Nature Genetics 18: 180-183).

In a particular embodiment, the viral vector for use in the methods of the present invention is an AAV vector. In particular embodiments, the viral vector is an AAV2/5 or AAV2/8 vector. Such vectors are described in, for example, U.S. Pat. No. 7,056,502, the entire contents of which are incorporated herein by reference. In another embodiment, adenoviral vectors suitable for use in the present invention may include those that are capable of transducing all retinal cell types upon intravitreal administration, such as an AAV2 variant having a V708I mutation. Additional suitable adenoviral vectors are those that do not generate a humoral immune response against the viral capsid upon administration (see, e.g., Dalkara et al, 2013 Sci Transl Med. 5, 189ra76), and those that facilitate nuclear transport of the AAV vector by, e.g., reducing ubiquitination and proteasome-mediated degradation of the vector, such as vectors having mutations that prevent phosphorylation of tyrosine residues in AAV capsid proteins (as described in e.g., Pang et al, 2010 The American Society of Gene & Cell Therapy. 19, 2: 234-242).

The vector will include one or more promoters or enhancers, the selection of which will be known to those skilled in the art. For example, commonly used promoters are derived from polyoma, adenovirus 2, cytomegalovirus and simian virus 40. Suitable promoters include, but are not limited to, the retroviral long terminal repeat (LTR), the SV40 promoter, the human cytomegalovirus (CMV) promoter, and other viral and eukaryotic cellular promoters known to the skilled artisan. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. In one embodiment, a tissue-specific promoter for use in the vectors and compositions of the invention is a retinal cell-specific promoter. In one embodiment, a retinal cell-specific promoter is a rod-, cone-, and bipolar cell-specific promoter. In one embodiment, a retinal cell-specific promoter is a rod- and cone-specific promoter. In one embodiment, a retinal cell-specific promoter is a rod-specific promoter. In one embodiment, a retinal cell-specific promoter is a cone-specific promoter.

Suitable retinal cell-specific promoters are known in the art and include, e.g., rhodopsin regulatory sequences, Nrl, Crx, Rax, and the like (Matsuda and Cepko (2007) Proc. Natl. Acad. Sci. U.S.A. 104:1027), opsin promoters, interphotoreceptor retinoid binding protein promoters (IRBP156), rhodopsin kinase (RK) promoters, neural leucine zipper (NRLL) promoters, (see, e.g., Semple-Rowland, et al. (2010) Molec Vision 16:916), or combinations thereof. additional suitable promoters may include Cabp5, Cralbp, Ndrg4, clusterin, Hesl, vimentin promoters, cluster differentiation (CD44) promoters, and glial fibrillary acid protein (GFAP) promoters.

Other suitable tissue-specific promoters that may be used in the compositions and methods of the invention include, for example, the albumin promoter (liver-specific, Pinkert et al. (1987) Genes Dev. 1:268), lymphoid-specific promoters (Calame and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. U.S.A. 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374) and the α-fetoprotein promoter (Campes and Tilghman (1989) Genes Dev. 3:537).

Guidance in the construction of gene therapy vectors and the introduction thereof into affected animals for therapeutic purposes may be obtained in the above-referenced publications, as well as in U.S. Pat. Nos. 5,631,236, 5,688,773, 5,691,177, 5,670,488, 5,529,774, 5,601,818, and PCT Publication No. WO 95/06486, the entire contents of which are incorporated herein by reference.

Generally, methods are known in the art for transfection and transformation of the cells of interest. For example, a virus can be placed in contact with the neuronal cell of interest or alternatively, can be injected into a subject suffering from a neurodegenerative disorder.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation (e.g., in vivo electroporation). Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals.

As discussed above, the nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470), stereotactic injection (see, e.g., Chen et al. (1994) Proc. Natl. Acad. Sci. U.S.A. 91:3054), or by in vivo electroporation (see, e.g., Matsuda and Cepko (2007) Proc. Natl. Acad. Sci. U.S.A. 104:1027). Local administration of nucleic acids and/or gene therapy vectors described herein can be by any suitable method in the art including, for example, injection (e.g., intravitreal or subretinal injection), gene gun, by topical application of the nucleic acid in a gel, oil, or cream, by electroporation, using lipid-based transfection reagents, transcleral delivery, by implantation of scleral plugs or a drug delivery device, or by any other suitable transfection method.

In one embodiment, a packaging cell line is transduced with a retroviral vector carrying the desired nucleic acid molecule to form a producer cell line. The packaging cells may be transduced by any means known in the art, including, e.g., electroporation, CaPO₄ precipitation, or the use of liposomes. Examples of packaging cells that may be transfected include, but are not limited to, BOSC23, Bing, PE501, PA317, .PSI.-2, .PSI.-AM, PA12, T19-14X, VT-19-17-H2, .PSI.-CRE, .PSI.-CRIP, GP+E86, GP+envAm12, and DAN cell lines. Guidance on retroviral producing packaging cells and how to construct them can be found in Short et al., J. Neurosci. Res. 27:427-433 (1990); Miller, A. D., Human Gene Ther. 1:5-14 (1990); Danos, 0, “Construction of Retroviral Packaging Cell Lines,” in Methods in Molecular Biology (M. Collins, ed.), Vol. 8, The Humana Press Inc., Clifton, N.J., 17-26 (1991); Murdoch, B., et al., Gene Therapy 4:744-749 (1997); and U.S. Pat. Nos. 5,529,774 and 5,591,624, the entire contents of which are incorporated herein by reference.

Retroviral vectors have may also be packaged with a vesicular stomatitis virus (VSV) envelope glycoprotein G (“pseudotyping”). These vectors are more stable and can be concentrated to 10⁹ cfu/ml, allowing them to be injected directly (Burns, J. C. et al. (1993) Proc. Natl. Acad. Sci. USA 90:8033-8037).

The producer cells can then be grafted near or into the desired location, for example, intraocularly. Direct injection of high titer retroviral producer cells (Murdoch, B., et al., Gene Ther. 4:744-749 (1997); Onodera, M., et al., Hum Gene Ther. 8:1189-1194 (1997)) allow for efficient in situ infection with the retroviral sequences (Rainov, N. G., et al., Cancer Gene Ther. 3:99-106 (1996); Ram, Z., et al., Cancer Res. 53:83-88 (1993)). Producer cells injected intraocularly do not generally migrate from the site of injection. Moreover, although they may be rejected by the host, this does not occur for 5-10 days, by which time retroviral infection of nearby cells will have occurred (Ram, Z., et al., J. Neurosurg. 79:400-407 (1993)). In general, vector producer cell (VPC) dosages range from about 2.5×10⁸ VPCs to about 1×10⁹ VPCs. The exact amount of producer cells will ultimately be determined by the skilled artisan based on numerous factors, including, but not limited to, the available injectable volume, clinical status of the patient, and the severity of the disorder.

Preferably, the viral genomes of the viral vectors used in the invention should be modified to remove or limit their ability to replicate, however, replication conditional viruses will also be useful in the present invention, as will replicating vectors that are capable of targeting certain cells. (See, e.g., Zhang, J. et al. (1996) Cancer Metastasis Rev. 15:385-401).

The nucleic acid molecules can also be delivered using non-viral methods for gene transfer, preferably those whose use in gene therapy is known in the art (Nakanishi, M., Crit. Rev. Therapeu. Drug Carrier Systems 12:263-310 (1995); Abdallah, B., et al., Biol Cell 85:1-7 (1995); Zhang, J., et al., Cancer Metastasis Rev. 15:385-401 (1996); Philips, S. C., Biologicals 23:13-16 (1995); Lee, R. J. and Huang, L., Crit. Rev. Ther. Drug Carrier Syst. 14:173-206 (1997)). Examples of such non-viral vectors for gene delivery include prokaryotic vectors, cationic liposomes, DNA-protein complexes, non-viral T7 autogene vectors (Chen, X., et al., Hum. Gene Ther. 9:729-736 (1998)), fusogenic liposomes, direct injection of nucleic acid (“naked DNA”), particle or receptor-mediated gene transfer, hybrid vectors such as DNA-adenovirus conjugates or other molecular conjugates involving a non-viral and viral component, starburstpolyamidoamine dendrimers (Kukowska-Latallo, J. F., et al., Proc Natl Acad Sci USA 93:4897-4902 (1996); Tang, M. X., et al., Bioconjug. Chem. 7:703-714 (1996)), cationic peptides (Wyman, T. B., et al., Biochemistry 36:3008-3017 (1997)), and mammalian artificial chromosomes (Ascenzioni, F., et al., Cancer Lett. 118:135-142 (1997)).

In addition, the present invention provides an embodiment of the foregoing methods wherein the nucleic acid molecules are delivered using any cellular vector, preferably one whose use for gene therapy is well-established for those skilled in the art. Examples of such cellular vectors for gene therapy include endothelial cells (Rancourt, C., et al., Clin. Cancer Res. 4:265-270 (1998); Qjeifo, J. O., et al., Cytokines Mol. Ther. 2:89-101 (1996)) and macrophages including tumor-infiltrating macrophages (Zufferey, R., et al., Nat. Biotechnol. 15:871-875 (1997); Naldini, L., et al., Science 272:263-267 (1996)), each of which may be modified using viral or non-viral vectors to carry the desired nucleic acid molecules, and thus express the desired gene products. Other suitable non-viral vectors will be readily apparent to the skilled artisan.

Gene delivery can be enhanced by including an internal ribosome entry site (IRES) sequence to achieve coordinate expression of multiple genes on a bicistronic message. IRESs are sequences containing 500-600 bp that are typical of the 5′ nontransduced regions of picornaviruses, including the polio- and encephalomyocarditis viruses (EMCV). See, e.g., Ghattas, I. R., et al., Molecular and Cellular Biology 11:5848-5859 (1991); Morgan, R. A., et al., Nucleic Acids Research 20:1293-1299 (1992). This approach has been used for efficient retroviral coexpression of the two subunits of interleukin-12 (Tahara, H., et al., J. Immunol. 154:6466-6474 (1995)). Another alternative is for the vector to contain multiple genes under the control of distinct promoters.

As discussed below, an isolated nucleic acid molecule of the invention and/or an isolated nucleic acid molecule of the invention within a vetor, e.g., a viral vector, e.g., for gene therapy purposes, may be prepared as a pharmaceutical composition. Such pharmaceutical preparations may include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Any suitable virus usable for nucleic acid delivery may be used, including, but not limited to, adenovirus, adeno-associated virus, retroviruses and the like. For example, the LIA retrovirus may be used to deliver nucleic acids (Cepko et al. (1998) Curr. Top. Dev. Biol. 36:51; Dyer and Cepko (2001) J. Neurosci. 21:4259). The viral titer may be varied to alter the expression levels. The viral titer may be in any suitable range. For example, the viral titer can have an upper limit of about 10⁵ cfu/ml, 10⁶ cfu/m, 10⁷ cfu/ml, 10⁸ cfu/ml, 10⁹ cfu/ml, 10¹⁰ cfu/ml, 10¹¹ cfu/ml or more. The viral titer can have a lower limit of about 10¹³ cfu/ml 10¹² cfu/ml, 10¹¹ cfu/ml, 10¹⁰ cfu/ml, 10⁹ cfu/ml, 10⁸ cfu/ml, 10⁷ cfu/ml, 10⁶ cfu/ml or less. Often, the viral titer ranges from about 10⁶ cfu/ml to 10⁸ cfu/ml. More often, the range is about 10⁷ cfu/ml to 10⁸ cfu/ml. The amount of virus to be added may also be varied. The volume of virus, or other nucleic acid and reagent, added can be in any suitable range. For example the volume may have an upper limit of about 100 μl, 200 μl, 300 μl, 400 μl, 500 μl, 750 μl, 1000 μl, 1250 μl, 1500 μl, or more. The volume may have a lower limit of about 1250 μl, 1000 μl, 750 μl, 500 μl, 400 μl, 300 μl, 200 μl, 100 μl, 50 μl, 25 μl, or less.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an HDAC4 peptide can be expressed in bacterial cells such as E. coli, viral cells such as retroviral cells, insect cells, yeast or mammalian cells (such as Chinese hamster ovary cells (CHO) or COS cells). Other suitable host cells are known to those skilled in the art.

The present invention also provides isolated HDAC4 peptides, e.g., N-terminal HDAC4 peptides that lack the HDAC4 enzymatic domain (e.g., deacetylase domain) and are derived from an HDAC4 amino acid sequence (e.g., the sequences disclosed herein as SEQ ID NOs.:2, 4, 6, 8, 10, and 12).

The isolated HDAC4 peptides of the invention can be made intracellularly in cells by introducing into the cells an expression vector encoding the peptide. Such expression vectors can be made by standard techniques. The peptide can be expressed in intracellularly as a fusion with another protein or peptide (e.g., a GST fusion). Alternative to recombinant synthesis of the peptides in the cells, the peptides can be made by chemical synthesis using standard peptide synthesis techniques. Synthesized peptides can then be introduced into cells by a variety of means known in the art for introducing peptides into cells (e.g., liposome and the like).

The isolated HDAC4 peptides may be localized to the cytoplasm of a neuronal cell, e.g., a retinal cell, inhibit neuronal, e.g., retinal, cell death, and/or are useful for, e.g., treating neurodegenerative disorders (described below). An HDAC4 peptide localized to the cytoplasm of a cell is predominately present in the cytoplasm with only minimal or no nuclear presence.

Accordingly, in one aspect, the present invention provides isolated peptides comprising amino acid residues 1-208 of SEQ ID NO:2. In one embodiment, the isolated peptides comprise amino acid residues 1-126 of SEQ ID NO:2. In another embodiment, the isolated peptides comprise amino acid residues 1-118 of SEQ ID NO:2. In yet another embodiment, the isolated peptides comprise amino acid residues 62-155 of SEQ ID NO:2.

In another aspect, the present invention provides isolated peptides comprising amino acid residues 1-207 of SEQ ID NO:4. In one embodiment, the isolated peptides comprise amino acid residues 1-125 of SEQ ID NO:4. In another embodiment, the isolated peptides comprise amino acid residues 1-117 of SEQ ID NO:4. In yet another embodiment, t the isolated peptides comprise amino acid residues 62-154 of SEQ ID NO:4.

In another aspect, the present invention provides isolated peptides comprising amino acid residues 1-230 of SEQ ID NO:6. In one embodiment, the isolated peptides comprise amino acid residues 1-148 of SEQ ID NO:6. In another embodiment, the isolated peptides comprise amino acid residues 1-140 of SEQ ID NO:6. In yet another embodiment, the isolated peptides comprise amino acid residues 84-177 of SEQ ID NO:6.

In another aspect, the present invention provides isolated peptides comprising amino acid residues 1-207 of SEQ ID NO:8. In one embodiment, the isolated peptides comprise amino acid residues 1-125 of SEQ ID NO:8. In another embodiment, the isolated peptides comprise amino acid residues 1-117 of SEQ ID NO:8. In yet another embodiment, the isolated peptides comprise amino acid residues 62-154 of SEQ ID NO:8.

In another aspect, the present invention provides isolated peptides comprising amino acid residues 1-208 of SEQ ID NO:10. In one embodiment, the isolated peptides comprise amino acid residues 1-126 of SEQ ID NO:10. In another embodiment, the isolated peptides comprise amino acid residues 1-118 of SEQ ID NO:10. In yet another embodiment, the isolated peptides comprise amino acid residues 62-155 of SEQ ID NO:10.

In another aspect, the present invention provides isolated peptides comprising amino acid residues 1-599 of SEQ ID NO:12. In one embodiment, the isolated peptides comprise amino acid residues 1-517 of SEQ ID NO:12. In another embodiment, the isolated peptides comprise amino acid residues 1-509 of SEQ ID NO:12. In yet another embodiment, the isolated peptides comprise amino acid residues 453-546 of SEQ ID NO:12.

### III. METHODS OF USE OF HISTONE DEACETYLASE COMPOSITIONS

As demonstrated in the Examples of the present application, the HDAC4 nucleic acid molecules of the invention (e.g., encoding HDAC4 peptides) inhibit neuronal cell death.

Accordingly, the present invention provides methods for inhibiting neuronal (e.g., retinal) cell death. Such methods generally comprise contacting a neuronal (e.g., retinal) cell with an HDAC4 nucleic acid molecule and/or peptide, as described herein. The present invention also provides methods for treating a subject having or prone to having a neurodegenerative disorder, e.g., an ocular disorder, e.g., age-related macular degeneration and retinitis pigmentosa. The methods generally comprise administering to a subject an effective amount (e.g., a prophylactically effective amount or a therapeutically effective amount) of an HDAC4 nucleic acid molecule and/or peptide, as described herein.

In certain embodiments of the invention, the nucleic acid molecule is contained within a vector, e.g., an expression vector, such as a retrovirus vector, an adenovirus vector, an adenoviral/retroviral chimera vector, an adeno-associated virus (AAV) vector, a herpes simplex virus I or II vector, a parvovirus vector, a reticuloendotheliosis virus vector, a poliovirus vector, a papillomavirus vector, a vaccinia virus vector, or a lentivirus vector. In one embodiment, the vector is an AAV vector. In one embodiment, the AAV vector is an AAV 2/5 or an AAV 2/8 vector. In other embodiments of the invention, the nucleic acid molecule is not contained within a vector.

One embodiment of the present invention involves a method for treatment of a neurodegenerative disorder, e.g., an ocular disorder, which includes the step of administering a therapeutically effective amount of an HDAC4 nucleic acid molecule (e.g., a gene therapy vector as described herein) to a subject.

In another embodiment, the present invention provides a method for treatment of a neurodegenerative disorder, e.g., ocular disorder, which includes the step of administering a therapeutically effective amount of an HDAC4 peptide to a subject.

An HDAC4 nucleic acid molecule and/or peptide as described herein may be administered to a subject having a neurodegenerative disorder, e.g., ocular disorder, or at risk of developing a neurodegenerative disorder, e.g., ocular disorder (e.g., a subject in which there is a family history of the neurodegenerative disorder, e.g., ocular disorder), prior to the onset of symptoms, such as loss of night-time vision due to, e.g., loss of rods. An HDAC4 nucleic acid molecule and/or peptide as described herein may also administered to a subject having a neurodegenerative disorder, e.g., ocular disorder, or at risk of developing a neurodegenerative disorder, e.g., ocular disorder (e.g., a subject in which there is a family history of the neurodegenerative retinal disorder), after the onset of symptoms, such as loss of night-time vision due to, e.g., loss of rods. In one embodiment, the cones of such a subject are viable.

In certain aspects of the invention, the neurodegenerative disorder, e.g., ocular disorder, is associated with decreased viability of cone cells. In other aspects of the invention, the neurodegenerative disorder, e.g., ocular disorder, is associated with decreased viability of rod cells. In still other aspects, the neurodegenerative disorder, e.g., ocular disorder, is a genetic disorder.

The principles of the present invention may be applied with particular advantage to treat, prevent and/or delay neuronal (e.g., retinal) cell loss by increasing the levels of an HDAC4 nucleic acid molecule expressed in a neuronal (e.g., retinal) cell.

In certain aspects, the levels of an HDAC4 peptide as described herein are altered, i.e., increased, in an organism to treat, prevent and/or delay neuronal (e.g., retinal) cell loss.

The principles of the present invention may also be applied to promote and/or accelerate neuronal (e.g., retinal) cell loss by decreasing the levels of an HDAC4 peptide expressed in a neuronal (e.g., retinal) cell. In certain aspects, the levels of an HDAC4 peptide are altered, i.e., decreased in an organism to treat aberrant proliferation of neuronal (e.g., retinal) cells by promoting and/or accelerating neuronal (e.g., retinal) cell loss.

For example, the methods of decreasing the levels of HDAC4 are useful to treat, prevent and/or delay one or more disorders and/or diseases associated with aberrant neuronal cell proliferation, e.g., cancer. Cellular proliferative disorders are intended to include disorders associated with rapid proliferation. As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995), incorporated herein by reference in its entirety for all purposes). The terms “neoplasm” and “tumor” refer to an abnormal tissue that grows by cellular proliferation more rapidly than normal. Id. Such abnormal tissue shows partial or complete lack of structural organization and functional coordination with the normal tissue which may be either benign (i.e., benign tumor) or malignant (i.e., malignant tumor). Examples of the types of neoplasms intended to be encompassed by the present invention include but are not limited to those neoplasms associated with cancers of neural tissue, blood forming tissue, breast, skin, bone, prostate, ovaries, uterus, cervix, liver, lung, brain, larynx, gallbladder, pancreas, rectum, parathyroid, thyroid, adrenal gland, immune system, head and neck, colon, stomach, bronchi, and/or kidneys.

In certain aspects, exogenous HDAC4 nucleic acid molecules of the invention are expressed in neuronal (e.g., retinal) cells such that total HDAC4 peptide levels are increased in the cell after exogenous HDAC4 expression when compared to total HDAC4 levels prior to exogenous HDAC4 expression. After exogenous expression of HDAC4, total HDAC4 levels can be increased by about 5%, 10%, 15%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000% or more over total HDAC4 levels prior to exogenous HDAC4 expression.

In other aspects, neuronal (e.g., retinal) cells are contacted with exogenous HDAC4 peptides, as described herein such that total HDAC4 peptide levels are increased in the cells after contacting the cells when compared to total HDAC4 levels prior to contacting the cells. After contacting the cells, total HDAC4 levels can be increased by about 5%, 10%, 15%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000% or more over total HDAC4 levels prior to contacting the cells.

In yet other aspects, neuronal (e.g., retinal) cells are contacted with exogenous HDAC4 nucleic acid molecules or HDAC4 peptides, as described herein, that decrease HDAC4 levels in the cell when compared to total HDAC4 levels prior to contact with the nucleic acid molecules HDAC4 peptides. After contact with the nucleic acid molecules or peptides, HDAC4 levels can be decreased to about 5%, 10%, 15%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000% or more over total HDAC4 levels prior to contact with the nucleic acid molecules or peptides.

As used herein, the term “administering” to a subject includes dispensing, delivering or applying a composition to a subject by any suitable route for delivery of the composition to the desired location in the subject, including delivery by intraocular (subretinal or subvitreal) administration, topical administration, transcleral administration or intravenous administration. Alternatively or in combination, delivery is by the parenteral or oral route, intracerebral injection, intramuscular injection, subcutaneous/intradermal injection, intravenous injection, buccal administration, transdermal delivery and administration by the rectal, colonic, vaginal, intranasal or respiratory tract route.

In general, the nucleic acid molecules and/or peptides of the invention and/or the vectors of the invention are provided in a therapeutically effective amount to elicit the desired effect, e.g., inhibit neuronal cell death. The quantity of the nucleic acid molecule, peptide, and/or vector to be administered, both according to number of treatments and amount, will also depend on factors such as the clinical status, age, and weight of the subject to be treated, and the severity of the disorder. Precise amounts of active ingredient required to be administered depend on the judgment of the gene therapist and will be particular to each individual patient. For example, a viral vector comprising the nucleic acid molecules of the invention is administered in titers ranging from about 1×10⁵ to about 1×10⁹ colony forming units (cfu) per ml, although ranges may vary. Preferred titers will range from about 1×10⁶ to about 1×10⁸ cfu/ml.

A therapeutically effective amount of the nucleic acid molecules, peptides, and/or the vectors of the invention (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of the nucleic acid molecules, peptides, and/or the vectors of the invention can include a single treatment or, preferably, can include a series of treatments. It will also be appreciated that the effective dosage used for treatment may increase or decrease over the course of a particular treatment. Changes in dosage may result from the results of diagnostic assays as described herein. The pharmaceutical compositions can be included in a container, pack, or dispenser together with instructions for administration.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Therapeutically effective amount,” as used herein, is intended to include the amount of a nucleic acid molecule, peptides, and/or the vectors of the invention that, when administered to a patient for treating a neurodegenerative disease, is sufficient to effect treatment of the disease (e.g., by diminishing, ameliorating or maintaining the existing disease or one or more symptoms of disease). The “therapeutically effective amount” may vary depending on the nucleic acid molecule, peptide, and/or the vector, how the nucleic acid molecule, peptide, and/or the vectorsis administered, the disease and its severity and the history, age, weight, family history, genetic makeup, stage of pathological processes mediated by the neurodegenerative disease expression, the types of preceding or concomitant treatments, if any, and other individual characteristics of the patient to be treated.

“Prophylactically effective amount,” as used herein, is intended to include the amount of a nucleic acid molecule, peptide, and/or the vector that, when administered to a subject who does not yet experience or display symptoms of a neurodegenerative disease, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the nucleic acid molecule, peptide, and/or the vector, how the nucleic acid molecule, peptide, and/or the vector is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of a nucleic acid molecule, peptide, and/or the vector that produces some desired local or systemic effect at a reasonable benefit/risk ratio applicable to any treatment. A nucleic acid molecule, peptide, and/or the vector employed in the methods of the present invention may be administered in a sufficient amount to produce a reasonable benefit/risk ratio applicable to such treatment.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

As used herein, the terms “treating” or “treatment” refer to a beneficial or desired result including, but not limited to, alleviation or amelioration of one or more symptoms, diminishing the extent of infection, stabilized (i.e., not worsening) state of infection, amelioration or palliation of the infectious state, whether detectable or undetectable. “Treatment” can also mean prolonging survival as compared to expected survival in the absence of treatment.

In certain embodiments of the invention, an HDAC4 nucleic acid molecule, peptide, and/or vector of the invention is administered in combination with an additional therapeutic agent or treatment. The compositions and an additional therapeutic agent can be administered in combination in the same composition or the additional therapeutic agent can be administered as part of a separate composition or by another method described herein.

Examples of additional therapeutic agents suitable for use in the methods of the invention include those agents known to treat neurodegenerative disorders, such as retinitis pigmentosa and age-related macular degeneration and include, for example, fat soluble vitamins (e.g., vitamin A, vitamin E, and ascorbic acid), calcium channel blockers (e.g., diltiazem) carbonic anhydrase inhibitors (e.g., acetazolamide and methazolamide), anti-angiogenics (e.g., antiVEGF antibodies), growth factors (e.g., rod-derived cone viability factor (RdCVF), BDNF, CNTF, bFGF, and PEDF), antioxidants, other gene therapy agents (e.g., optogenetic gene threrapy, e.g., channelrhodopsin, melanopsin, and halorhodopsin), and compounds that drive photoreceptor regeneration by, e.g., reprogramming Müller cells into photoreceptor progenitors (e.g., alpha-aminoadipate). Exemplary treatments for use in combination with the treatment methods of the present invention include, for example, retinal and/or retinal pigmented epithelium transplantation, stem cell therapies, retinal prostheses, laser photocoagulation, photodynamic therapy, low vision aid implantation, submacular surgery, and retinal transloacation.

### IV. PHARMACEUTICAL COMPOSITIONS

In one aspect of the invention, a therapeutic nucleic acid molecule, peptide, and/or vector containing the same will be in the form of a pharmaceutical composition containing a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. In one embodiment, the pharmaceutically acceptable carrier is not phosphate buffered saline (PBS). In one embodiment, the carrier is suitable for intraocular, topical, parenteral, intravenous, intraperitoneal, or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the gene therapy vector, use thereof in the pharmaceutical compositions of the invention is contemplated. Supplementary active compounds can also be incorporated into the compositions.

In a particular embodiment, the pharmaceutical compositions of the present invention would be administered in the form of injectable compositions. The compositions can be prepared as an injectable, either as liquid solutions or suspensions. The preparation may also be emulsified. Suitable excipients are, for example, water, saline, dextrose, glycerol, ethanol, or the like and combinations thereof. In addition, if desired, the preparation may contain minor amounts of auxiliary substances such as wetting or emulsifying agents, pH-buffering agents, adjuvants or immunopotentiators.

In a particular embodiment, the nucleic acid molecules and/or vectors are incorporated in a composition suitable for intraocular administration. For example, the compositions may be designed for intravitreal, subconjuctival, sub-tenon, periocular, retrobulbar, suprachoroidal, and/or intrascleral administration, for example, by injection, to effectively treat the retinal disorder. Additionally, a sutured or refillable dome can be placed over the administration site to prevent or to reduce “wash out”, leaching and/or diffusion of the active agent in a non-preferred direction.

Relatively high viscosity compositions, as described herein, may be used to provide effective, and preferably substantially long-lasting delivery of the nucleic acid molecules and/or vectors, for example, by injection to the posterior segment of the eye. A viscosity inducing agent can serve to maintain the nucleic acid molecules and/or vectors in a desirable suspension form, thereby preventing deposition of the composition in the bottom surface of the eye. Such compositions can be prepared as described in U.S. Pat. No. 5,292,724, the entire contents of which are hereby incorporated herein by reference.

Sterile injectable solutions can be prepared by incorporating the compositions of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: A binder such as microcrystalline cellulose, gum tragacanth or gelatin; an excipient such as starch or lactose, a disintegrating agent such as alginic, acid, Primogel, or corn starch; a lubricant such as magnesium stearate or Sterotes; a glidant: such as colloidal silicon dioxide; a sweetening agent such as sucrose or saccharin; or a flavoring agent such as peppermint, methyl salicylate, or orange flavoring.

In one embodiment, the compositions of the invention are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811.

Nasal compositions generally include nasal sprays and inhalants. Nasal sprays and inhalants can contain one or more active components and excipients such as preservatives, viscosity modifiers, emulsifiers, buffering agents and the like. Nasal sprays may be applied to the nasal cavity for local and/or systemic use. Nasal sprays may be dispensed by a non-pressurized dispenser suitable for delivery of a metered dose of the active component. Nasal inhalants are intended for delivery to the lungs by oral inhalation for local and/or systemic use. Nasal inhalants may be dispensed by a closed container system for delivery of a metered dose of one or more active components.

In one embodiment, nasal inhalants are used with an aerosol. This is accomplished by preparing an aqueous aerosol, liposomal preparation or solid particles containing the compound. A non-aqueous (e.g., fluorocarbon propellant) suspension could be used. Sonic nebulizers may be used to minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compositions of the invention can also be prepared in the form of suppositories (e.g., with conventional suppository bases such as cocoa butter and other glycerides) or retention enemas for rectal delivery.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of nucleic acid molecules described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and/or animal studies can be used in formulating a range of dosage for use in humans. The dosage typically will lie within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, and accompanying claims.

### EXAMPLES

### Example 1

### Photoreceptor Cell Rescue by HDAC4 Gene Therapy

Previous work has demonstrated that electroporation of rods in a mouse model of retinitis pigmentosa (RP) at postnatal day 0 (P0) with full-length histone deacetylase 4 (HDAC4), including the enzymatic deacetylase domain, preserved rod survival. Indirectly, due to the lack of rod death, cones also showed greater survival (Chen B. and Cepko C. L. (2009) Science 9:323(5911):256-9; U.S. Patent Publication No. 2011/0268705, the entire contents of which are incorporated herein by reference).

Since electroporation does not transduce all cells, and since it may not be a suitable method for long term transduction, there is a need for alternative means of gene transfer. In particular, gene therapy directed to the photoreceptor cells can produce broader and more stable transduction. The use of gene therapy viral vectors also has the advantage of being able to transduce a wider range of species than electroporation. In addition, gene therapy directed to the photoreceptor cells enables a steady level of the agent in the retina, and reduces side effects associated with systemic exposure since it bypasses the blood-retinal barrier.

Gene therapy using adeno-associated viral (AAV) vectors have shown great promise in human clinical trials of Leber's congenital amaurosis (LCA), a disease with early onset leading to complete blindness (Bainbridge, J. W., et al. (2008) N Engl J Med 358:2231-2239; Cideciyan, A. V., et al. (2009) Human Gene Therapy 20:999-1004; Maguire, A. M., et al. (2008) N Engl J Med 358:2240-2248).

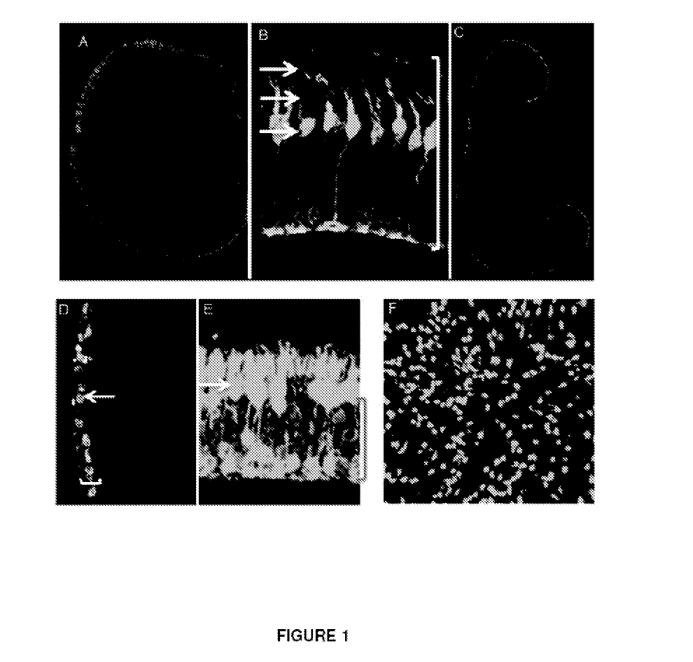

AAV vectors can infect both rods and cones very effectively. For example, as depicted in FIG. 1, infection of retinas with an AAV vector results in high levels of gene expression for long periods of time. Indeed, in dogs treated with an AAV vector, the expression persisted for at least 10 years when the injections were made into puppies (Acland, G. M., et al. (2001) Nature Genetics 28:92-95).

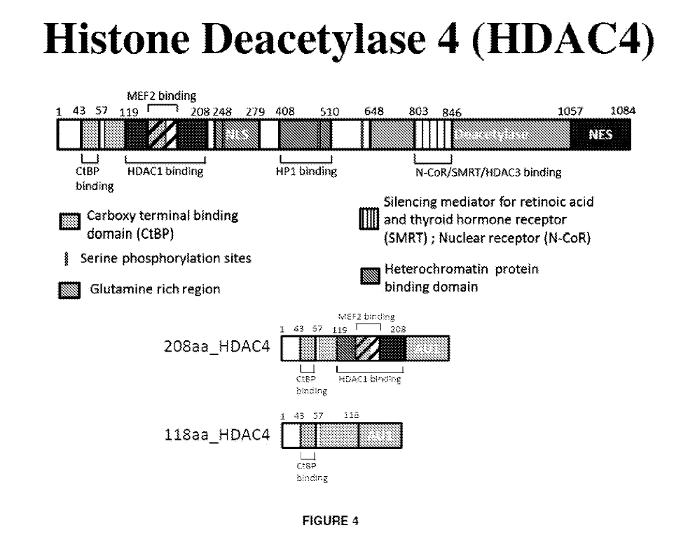

HDAC4 is a large protein (see, e.g., FIG. 4) having multiple binding domains. In order to increase the efficiency of transfection of photoreceptor cells with an AAV vector comprising the HDAC4 coding sequence and to reduce any potential off-target effect, fragments of HDAC4 were tested for their ability to inhibit death of rod cells by electroporating the retinas of rd1 mice having RP (Bowes, C., et al. (1990) Nature 347:677-680).

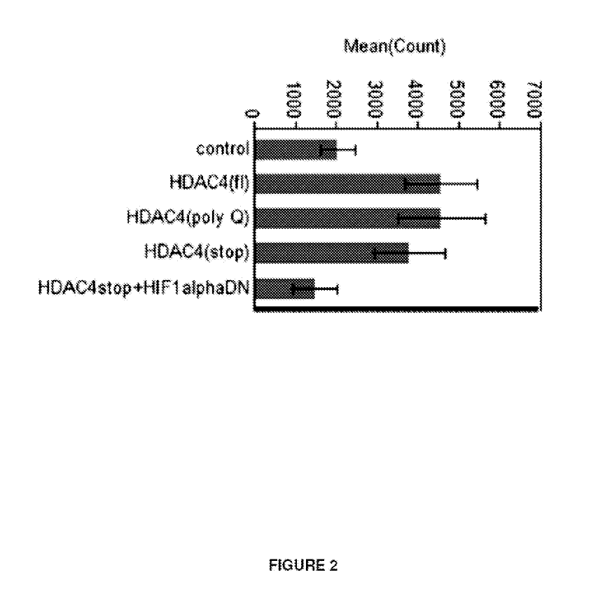

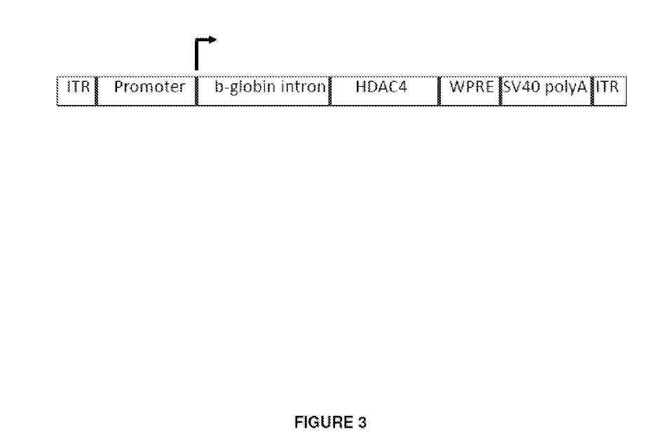

The results of these experiments are depicted in FIG. 2 and surprisingly demonstrate that only a small N-terminal portion of HDAC4 lacking its deacetylase domain promoted rod survival.

In order to determine which domains of the N-terminal portion of HDAC4 are necessary and sufficient to rescue rods in an animal model of RP, additional deletion alleles of HDAC4 were generated, inserted into a plasmid vector and electroporated into the retinas of rd mice.

In particular, vectors comprising the CAG promoter driving expression of full length HDAC4 (FL; amino acid residues 1-1084 of SEQ ID NO:2), or amino acid residues 1-118 of SEQ ID NO:2 (118aaAU1), or amino acid residues 1-208 of SEQ ID NO:2 (208aaAU1), and an epitope to allow tracking of the protein (AU1) were electroporated into the retinas of rd mice at postnatal day 1. Two additional plasmids were also electroporated into the retinas of these mice, CAG-GFP and Rho=DsRed. The CAG-GFP construct was used as a marker of the area of the retina that was electroporated and the Rho-DsRed construct was used as a marker of rod cells.

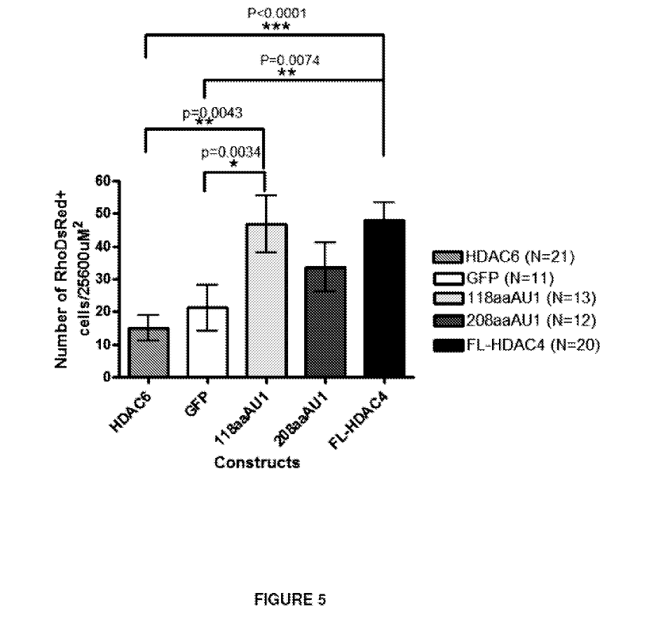

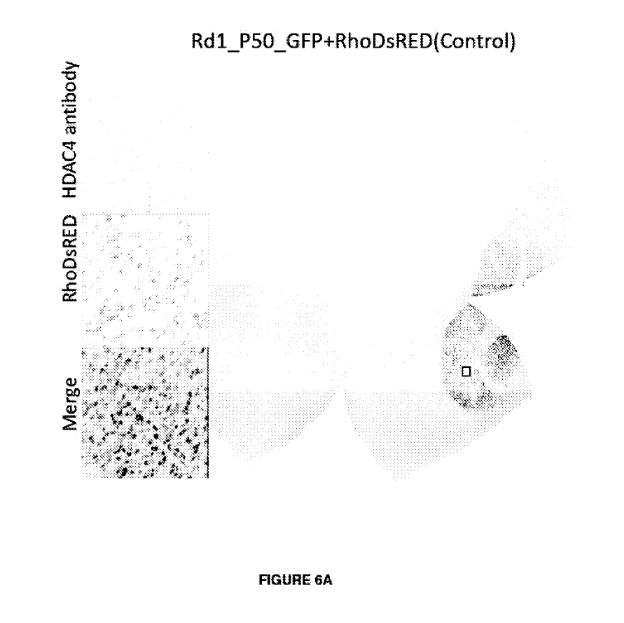

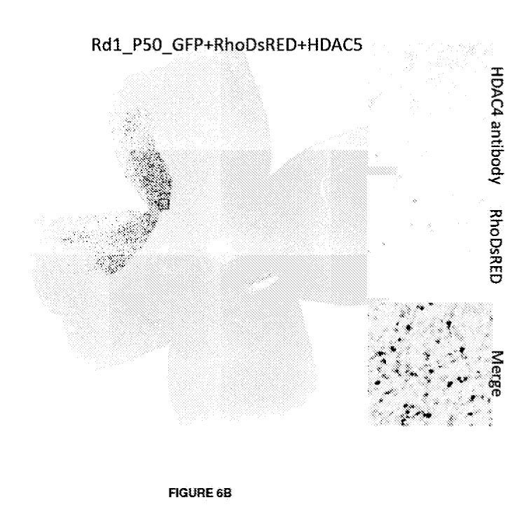

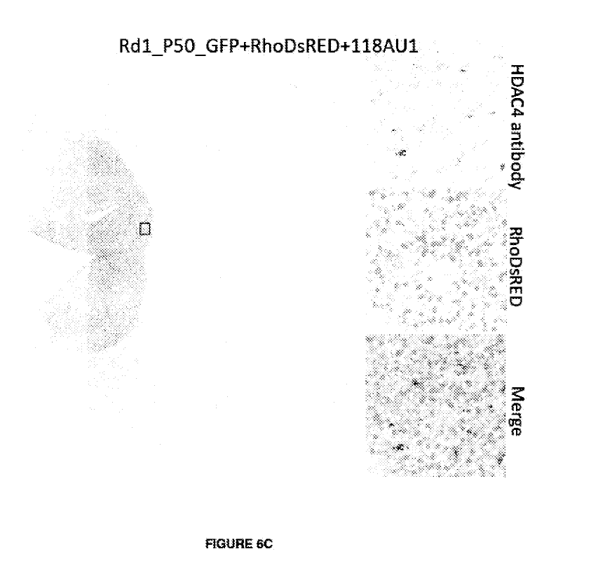

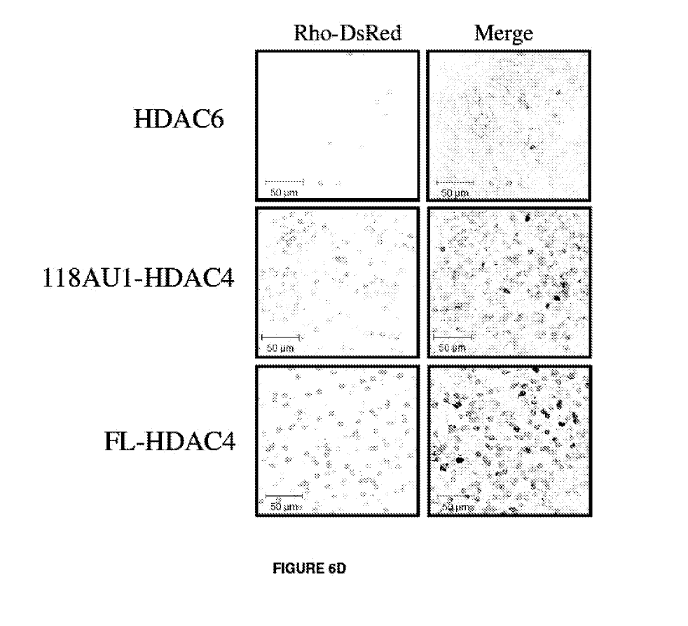

At postnatal day 50, the cells that were positive for Ds-Red in the electroporated area (CAG-GFP positive) were counted in a domain of 25, 600 microns. As shown in FIGS. 5 and 6, both N-terminal constructs, 118aaAU1 and 208aaAU1, promoted rod survival. Moreover, the results surprisingly demonstrate that neither the deacetylase domain of HDAC4 nor the domains of HDAC4 necessary for interaction with its known partners, Mef2, HDAC1, HP1, and HDAC3 (see FIG. 4), are necessary for promoting rod survival. These short nucleic acid molecules encoding these small peptides will easily fit into a vector, such as a viral vector, and will have minimal off-target effects which result from interaction with other proteins in the cell. Accordingly, nucleic acid molecules encoding these short HDAC4 peptides may be inserted in an adenoviral or lentiviral vector, such as an AAV vector useful for gene therapy. Such vectors may include promoters that specifically express the HDAC4 nucleic acid molecules, in cones, rods, cones and rods, and/or cones, rods, and bipolar cells. Retinas are infected with the vectors using suitable methods and tested for their ability to inhibit rod and cone death in suitable animal models, such as mouse models of retinitis pigmentosa, pig models of retinitis pigmentosa, and/or dog models of retinitis pigmentosa (see, e.g., Rossmiller, et al. (2012) Molec Vision 18:2479 for a review).

Survival and function of rods and cones may be quantified using standard histological techniques, such as those described herein. Behavioral assays for vision (Pearce-Kelling, S. E., et al. (2001) Molecular vision 7:42-47), and electrophysiological analysis of signals from rods and cones using an electroretinogram (ERG), or via recording from the ganglion cells may also performed on treated animals (Lagali, P. S., et al. (2008) Nature Neuroscience 11:667-675).

1. A pharmaceutical composition suitable for intraocular administration, comprising
(a) an isolated HDAC4 nucleic acid molecule comprising nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1;
(b) an isolated HDAC4 nucleic acid molecule encoding a peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2; or
(c) an isolated HDAC4 peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2.

2. (canceled)

3. (canceled)

4. A composition comprising a viral vector comprising a retinal cell-type specific promoter operably linked to
(a) an HDAC4 nucleic acid molecule comprising nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1; or
(b) a nucleic acid molecule encoding an HDAC4 peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2.

5. (canceled)

6. The composition of claim 4, wherein the retinal cell-type specific promoter is a rod-specific promoter, a cone-specific promoter, or a rod- and cone-specific promoter.

7.-9. (canceled)

10. A method of inhibiting neuronal cell death, comprising contacting said cell with
(a) an isolated HDAC4 nucleic acid molecule comprising nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1;
(b) an isolated nucleic acid molecule encoding an HDAC4 peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2; or
(c) an isolated HDAC4 peptide comprising amino acid residues 1-208 of SEQ ID NO:2□ amino acid residues 1-126 of SEQ ID NO:2, amino acid residues 1-118 of SEQ ID NO:2, or amino acid residues 62-155 of SEQ ID NO:2, thereby inhibiting death of the neuronal cell.

11. A method for treating or preventing a neurodegenerative disorder in a subject comprising administering to said subject
(a) an isolated HDAC4 nucleic acid molecule comprising nucleotides 793-1416 of SEQ ID NO:1, nucleotides 793-1170 of SEQ ID NO:1, nucleotides 793-1146 of SEQ ID NO:1, or nucleotides 978-1257 of SEQ ID NO:1;
(b) an isolated nucleic acid molecule encoding an HDAC4 peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2; or
(c) an isolated HDAC4 peptide comprising amino acid residues 1-208 of SEQ ID NO:2, amino acid residues 1-126 of SEQ ID NO:2, amino acid residues 1-118 of SEQ ID NO:2, or amino acid residues 62-155 of SEQ ID NO:2, thereby treating or preventing the neurodegenerative disorder in the subject.

12.-15. (canceled)

16. The method of claim 10 or 11, wherein the nucleic acid molecule encodes a peptide localized to the cytoplasm of the neuronal cell or the peptide is localized to the cytoplasm of the neuronal cell.

17. (canceled)

18. (canceled)

19. The method of claim 10, wherein the cell is a retinal cell.

20. The method of claim 19, wherein the retinal cell is a bipolar cell, a rod photoreceptor cell and/or a cone photoreceptor cell.

21. (canceled)

22. (canceled)

23. The method of claim 10, wherein the neuronal cell death is naturally occurring.

24. The method of claim 10, wherein the neuronal cell death is caused by a neurodegenerative disorder.

25. The method of claim 11 or 24, wherein the neurodegenerative disorder is age-related macular degeneration.

26. The method of claim 11 or 24, wherein the neurodegenerative disorder is retinitis pigmentosa.

27.-31. (canceled)

32. The method of claim 10, wherein the contacting is within the eye of a subject.

33. The method of claim 11, wherein the administration is intraocular administration.
